(19) United States
(12) Patent Application Publication
(10) Pub. No.: US 2026/0094717 A1
(43) Pub. Date: Apr. 2, 2026

(54) NON-INVASIVE BONE MARROW DIAGNOSTICS

(71) Applicant: YEDA RESEARCH AND DEVELOPMENT CO. LTD., Rehovot (IL)

(72) Inventors: Liran SHLUSH, Herzeliya (IL); Amos TANAY, Rehovot (IL)

(21) Appl. No.: 19/411,342

(22) Filed: Dec. 7, 2025

Related U.S. Application Data
(63) Continuation of application No. PCT/IL2024/050568, filed on Jun. 9, 2024.

(30) Foreign Application Priority Data
Jun. 8, 2023 (IL) 303582

(51) Int. Cl. G16H 50/20; G16B 30/10; G16B 40/20; G16H 20/10; G16H 50/30

(52) U.S. Cl. CPC G16H 50/20; G16B 30/10; G16B 40/20; G16H 20/10; G16H 50/30

(57) ABSTRACT

Non-invasive methods of detecting pathology of the bone marrow comprising receiving a metacell model of a plurality of metacell types based on single cell RNA sequencing (scRNA-seq) of CD34 positive cells from peripheral blood and comparing it to control values of metacells of CD34 positive cells from peripheral blood of healthy subjects are provided. Non-invasive methods of predicting the percentage of blasts in the bone marrow and of calculating an IPSS-M risk score are also provided, as are systems for performing the methods of the invention.

### CROSS REFERENCE TO RELATED APPLICATIONS

This application is a ByPass Continuation of PCT Patent Application No. PCT/IL2024/050568 having International filing date of Jun. 9, 2024, which claims the benefit of priority of Israeli Patent Application No. 303582, filed Jun. 8, 2023, the contents of which are all incorporated herein by reference in their entirety.

### FIELD OF INVENTION

The present invention is in the field of bone marrow diagnostics.

### BACKGROUND OF THE INVENTION

The basis for understanding and defining human pathophysiological states is a detailed description of inter-individual heterogeneity among healthy individuals. Variability between healthy humans is multifactorial and determined by the interaction between germline/somatic mutations and the environment. The identification of inter-individual changes in complete blood counts (CBC) in large cohorts of healthy individuals exposed different age-related deviations from the reference. Such studies uncovered age-related macrocytic anemia with increased RDW and a reduction in absolute lymphocyte counts. The mechanisms responsible for both phenomena remain enigmatic. Another aspect of heterogeneity in the blood is the appearance of somatic mutations in hematopoietic stem and progenitor cells (HSPCs). All HSPCs acquire somatic mutations, however, certain mutations in leukemia-related genes, namely pre-leukemic mutations—pLMs, can lead to clonal expansion of HSPCs, a phenomenon termed clonal hematopoiesis (CH). While CH is quite common among the elderly, it remains poorly understood why pLMs lead to clonal expansion, and how CH and other age-related blood phenomena are related to each other.

One of the major gaps for understanding these age-related phenomena in the blood is our insufficient knowledge of HSPC variability across healthy, age-diverse individuals. While the various HSPC subpopulations and their functions have been extensively studied, it remains poorly understood how these differ between individuals. Inter-individual heterogeneity in the frequency of CD34+ peripheral blood (PB) HSPCs has been reported in the past, and was linked to age, smoking, sex, and hereditary factors, as well as different pathological states. Some studies analyzed HSPC heterogeneity in higher resolution, but their sample size was limited. No study specifically determined the inter-individual heterogeneity in HSPC transcriptional programs in a large cohort of healthy individuals, and how these correlated with CBC, CH and age.

Such a reference map has not yet been described, as the tools to characterize transcriptional programs in HSPCs with minimal bias, and at single cell resolution, have just been recently developed. In addition, as most HSPCs reside within the bone marrow (BM), access to these cells, in particular from healthy donors, has been problematic. However, previous studies have demonstrated that most HSPC populations can be identified in the PB, including some based on scRNAseq analysis, and functional stem cells were identified in the PB of mice and humans. As the PB connects the BM to other extramedullary stem cell sites, it can be enriched in unique stem cell populations. All this suggests that PB HSPCs can be a good surrogate for studying inter-individual HSPC transcriptional heterogeneity. A new accurate, non-invasive test for assessing MSPCs of the bone marrow by examining HSPCs in PB therefore greatly needed.

### SUMMARY OF THE INVENTION

The present invention provides non-invasive methods of detecting pathology of the bone marrow comprising receiving a subject cellular dataset based on single cell RNA sequencing (scRNA-seq) of CD34 positive cells from peripheral blood of the subject and analyzing the received subject cellular dataset in relation to a control dataset comprising a plurality of cellular datasets wherein each cellular dataset of the plurality is based on scRNA-seq of CD34 positive cells from peripheral blood of a healthy subject. Non-invasive methods of predicting the percentage of blasts in the bone marrow and of calculating an IPSS-M risk score are also provided, as are systems for performing the methods of the invention.

According to a first aspect, there is provided a non-invasive method of detecting pathology of the bone marrow in a subject in need thereof, the method comprising:
a. receiving a subject cellular dataset based on single cell RNA sequencing (scRNA-seq) of CD34 positive cells from peripheral blood of the subject; and
b. analyzing the received subject cellular dataset in relation to a control dataset comprising a plurality of cellular datasets wherein each cellular dataset of the plurality is based on scRNA-seq of CD34 positive cells from peripheral blood of a healthy subject, wherein a deviation of the subject cellular dataset from the control dataset indicates a bone marrow pathology;
   thereby detecting pathology of the bone marrow.

According to some embodiments, the cellular dataset comprises statistical data of the totality of CD34 positive cells in a peripheral blood sample.

According to some embodiments, the analyzing comprises producing a feature vector representing deviation of the subject's cellular data from the control cellular data.

According to some embodiments, the analyzing comprises applying a trained machine learning model to the received dataset, wherein the machine learning model is trained on a training set comprising the plurality of cellular datasets and wherein the machine learning model classifies the subject's bone marrow as being a healthy or not.

According to some embodiments, the training set further comprises cellular datasets based on scRNA-seq of CD34 positive cells from peripheral blood of subjects suffering from pathology of the bone marrow and labels indicating a cellular dataset is from a healthy subject or a subject with pathology of the bone marrow; and wherein the machine learning model classifies the subject as being heathy or suffering from a pathology of the bone marrow.

According to some embodiments, the analyzing comprises applying a trained machine learning model to the feature vector, wherein the machine learning model is trained on a training set comprising: feature vectors from healthy subjects and subjects suffering from pathology of the bone marrow and labels indicating a feature vector is from a healthy subject or a subject with pathology of the bone marrow; and wherein the machine learning model classifies the subject as being heathy or suffering from a pathology of the bone marrow.

According to some embodiments, the analyzing comprises applying a trained machine learning model to a parameter extracted from the cellular dataset, wherein the machine learning model is trained on a training set comprising: the parameter extracted from cellular datasets of healthy subjects and optionally subjects suffering from a bone marrow pathology and wherein the machine learning model classifies the subject as being a healthy subject or not.

According to some embodiments, the cellular dataset is selected from: a metacell model of the totality of CD34 positive cells in a peripheral blood sample, a transcriptome of each of the CD34 positive cells in a peripheral blood sample, an annotated cell atlas of CD34 positive cell types present in a peripheral blood sample.

According to some embodiments, the pathology of the bone marrow is selected from myelodysplastic syndrome (MDS), Chronic myelomonocytic leukemia (CMML), Acute myeloid leukemia (AML), polycythemia vera (PV), essential thrombocythemia (ET), Mastocytosis, chronic eosinophilic leukemia, myelofibrosis (MF), acute lymphoblastic leukemia (ALL), acute leukemia of ambiguous lineage, multiple myeloma (MM), myeloproliferative neoplasm (MPN) and blastic plasmacytoid dendritic cell leukemia.

According to some embodiments, the method is a method of detecting MDS and wherein deviation in the frequency of erythrocyte progenitor cells (ERYP), basophil/eosinophil/mast progenitor cells (BEMP), and/or megakaryocyte/erythrocyte/basophil/eosinophil/mast progenitor cells (MEBEMP) indicates the presences of MDS.

According to some embodiments, the method is a method of detecting CMML and wherein deviation in the frequency of early granulocyte-monocyte progenitor cells (GMP-E) indicates the presence of CMML.

According to some embodiments, the method is a method of detecting AML and wherein deviation in the frequency of common lymphoid progenitor cells (CLP) and/or natural killer/T/dendritic cell progenitor cells (NKTDP) indicates the presence of AML.

According to some embodiments, the deviation is higher or lower levels of a cell types than is present in the healthy subjects.

According to some embodiments, deviation in the frequency of CLPs is also indicative of MDS and wherein the deviation is lower levels of the CLPs than is present in the healthy subjects.

According to some embodiments, deviation in the frequency of CLPs is also indicative of CMML, MF or MPN and wherein the deviation is lower levels of the CLPs than is present in the healthy subjects.

According to some embodiments, the method is a method of detecting MDS and wherein a decrease in the frequence of CLP, NKTDP or both as compared to healthy subjects is indicative of MDS.

According to some embodiments, a decrease in the frequency of both CLP and NKTDP as compared to healthy subjects is indicative of MDS.

According to some embodiments, the pathology of the bone marrow comprises an increased percentage of blasts, wherein deviation is an increase and wherein a deviation in the frequency of early common lymphoid progenitor cells (CLP-E) indicates the presence of an increased percentage of blasts.

According to some embodiments, the method further comprises administering at least one therapeutic agent to a subject determined to suffer from a bone marrow pathology.

According to another aspect, there is provided a non-invasive method of predicting the percentage of blasts in the bone marrow of a subject in need thereof, the method comprising receiving a measure of the CLP-E cells in the peripheral blood of the subject wherein the measure is proportional to the percentage of blasts in the bone marrow of the subject, thereby predicting the percentage of blasts in the bone marrow of a subject.

According to some embodiments, the method further comprises analyzing the received measure in relation to a control dataset comprising a plurality of measures of CLP-E cells in the peripheral blood of healthy subjects and subjects suffering from pathology of the bone marrow, wherein the percentage of blasts in the bone marrow is known for each subject of the control dataset.

According to another aspect, there is provided a non-invasive method of predicting the percentage of blasts in the bone marrow of a subject in need thereof, the method comprising:
a. receiving a subject cellular dataset based on single cell RNA sequencing (scRNA-seq) of CD34 positive cells from peripheral blood of the subject; and
b. applying a trained machine learning model to the received dataset, wherein the machine learning model is trained on a training set comprising a plurality of cellular datasets wherein each cellular dataset of the plurality is based on scRNA-seq of CD34 positive cells from peripheral blood of a control subject and labels indicating the percentage of blasts in the bone marrow of the control subjects that provided each cellular dataset of the plurality of cellular datasets; and wherein the machine learning model outputs a predicted percentage of blasts in the bone marrow of the subject;
   thereby predicting the percentage of blasts in the bone marrow of a subject.

According to some embodiments, the subject suffers from leukemia.

According to some embodiments, the control subjects comprise subjects suffering from leukemia and non-leukemic subjects.

According to some embodiments, the cellular dataset is selected from: a metacell model of the totality of CD34 positive cells in a peripheral blood sample, a transcriptome of each of the CD34 positive cells in a peripheral blood sample, an annotated cell atlas of CD34 positive cell types present in a peripheral blood sample.

According to some embodiments, the cellular data set is a metacell model and is produced by a method comprising:
a. receiving a peripheral blood sample from a subject;
b. isolating CD34 positive hematopoietic stem and progenitor cells (HSPCs) from the peripheral blood sample;
c. performing scRNA-seq of the isolated HSPCs to produce a transcriptome for each isolated HSPC; and
d. producing a metacell model of the HSPCs based on their transcriptomes.

According to some embodiments, a metacell is a cluster of cells with a similar transcriptome.

According to some embodiments, a cellular dataset comprises groupings of cells into cell types that share a common differentiation within the HSPC spectrum of differentiation.

According to some embodiments, the cell types are selected from: BEMP, ERYP, MEBEMP-L, MEBEMP-E, GMP-E, multipotent progenitor cells (MPP), hematopoietic stem cells (HSC), CLP-E, CLP-M, CLP-L and NKTDP.

According to some embodiments, the method is a method of detecting MDS and/or leukemia and wherein a percentage of blasts above a predetermined threshold indicates the subject suffers from MDS and/or leukemia.

According to some embodiments, the method further comprises administering to a subject suffering from MDS and/or leukemia at least one anticancer therapy.

According to another aspect, there is provided a non-invasive method of calculating a Molecular International Prognostic Scoring System (IPSS-M) risk score for a subject suffering from a bone marrow malignancy, the method comprising:
a. predicting the percentage of blasts in the bone marrow of the subject by a method of the invention;
b. detecting the presence of bone marrow mutations and karyotype abnormalities based on scRNA-seq reads from CD34 positive cells from peripheral blood of the subject;
c. receiving hemoglobin levels, and platelet counts in peripheral blood from the subject; and
d. calculating the IPSS-M risk score based on the predicted blast percentage, detected mutations and karyotyping and received hemoglobin levels and platelet counts;
   thereby calculating an IPSS-M risk score.

According to some embodiments, the method further comprises administering to the subject a treatment regimen based on the IPSS-M risk score, where in a subject with a higher score is administered a more intense treatment regimen and a subject with a lower score is administered a reduced treatment regimen.

According to another aspect, there is provided a system for evaluating bone marrow health in a subject, the system comprising:
a scRNA sequencing device;
a non-transitory memory device, wherein modules of instruction code are stored;
and at least one processor associated with the memory device, and configured to execute the modules of instruction code, whereupon execution of the modules of instruction code, the at least one processor is configured to:
obtain from the scRNA sequencing device single cell transcriptomes from CD34 positive cells from peripheral blood of the subject;
produce a cellular dataset based on the obtained single cell transcriptomes;
analyze the produced cellular dataset in relation to a control dataset comprising a plurality of cellular datasets wherein each cellular dataset of the plurality is based on scRNA-seq of CD34 positive cells from peripheral blood of a healthy subject and
output a finding of healthy bone marrow or pathology of the bone marrow in the subject based on deviation of the subject cellular dataset from the control dataset.

According to some embodiments, the cellular dataset is a metacell model with similar transcriptomes from the obtained single cell transcriptomes clustered into metacells.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing(s) executed in color. Copies of this patent or patent application publication with color drawing(s) will be provided by the office upon request and payment of the necessary fee.

FIGS. 1A-1H: (1A) experimental design. (1B) annotated 2D UMAP projection of the metacell manifold following filtration of metacells with low CD34 expression. (1C-D) (1C) Symmetric and (1D) asymmetric regulation of specific HSC transcription factors upon bifurcation to the CLP (right) and MEBEMP (left) lineages. Each panel shows the expression of one gene (Y axis). Metacells in all panels are ordered (left to right) by increasing AVP expression in the MEBEMP lineage and decreasing AVP expression in the CLP lineage. Units for gene expression in all the figure panels are log2 of each gene's fractional expression. (1E) the metacell population of interest (dotted line) linking BEMPs to their MEBEMP-L precursors. (1F) positively and negatively regulated TFs involved in early BEMP differentiation. (1G) gene-gene plot of IRF8 against TCF7 expression as hallmark markers of DC and T cell differentiation respectively. The high ACY3 NKTDP metacell population of interest is depicted (dotted line). (1H) This population exhibits high expression of both T and dendritic cell regulators, forming a gradient consisting of NK/T cell-like progenitors exhibiting a high TCF7/IRF8 expression ratio along with high expression of other T cell hallmarks such as CD7, MAF, IL7R, TRBC2, and DC-like progenitors exhibiting a low TCF7/IRF8 expression ratio, along with high expression of other DC hallmarks, such as the myeloid TF PU.1 and the MHC class II gene CD74.

FIGS. 2A-2H: (2A) characterization of inter-individual HSPC compositional state variation (scheme). (2B) boxplots of cell state frequency distributions across individuals (logarithmic scale). Percents calculated out of CD34+ population. Boxplot centers, hinges and whiskers represent median, first and third quartiles and 1.5× interquartile range, respectively. Numbers represent mean +/−SD for each distribution. (2C) correlation of cell state frequencies between 20 biological replicates and their original samples, for CLP (CLP-E, CLP-M, CLP-L, NKTDP—top) and MEBEMP (MEBEMP-E, MEBEMP-L, ERYP, BEMP-bottom) populations. All biological replicates were sampled 1 year following original blood draw. (2D) (top)—individual cell state frequency profiles over the HSC-MEBEMP and HSC-CLP differentiation gradients of 6 subjects (colored lines), each representing one of six archetypes (classes) of HSPC composition in healthy individuals. Dashed lines represent the median (black) and 5th and 95th percentiles (grey) of the studied population. (bottom) cell state enrichment map over 15 differentiation bins (rows), for all studied individuals (columns) clustered into 6 classes. Classes I & II represent individuals relatively enriched in lymphoid progenitors, whereas classes V & VI represent individuals with relative depletion of lymphoid progenitors. Individuals are sorted by stemness in each class. Age and sex are denoted for each individual. (2E) CBC correlations to cell type frequencies: % Lymphocytes (from WBC, calculated for entire cohort, left), HCT (males only, center), RDW (males only, right). Missing individuals lacked sufficient cells for analysis. Permutation test p values are displayed for each correlation. See Methods for details on the permutation-based test. (2F) boxplots of CLP frequency distributions in individuals with (right) and without (left) clonal hematopoiesis. (2G) Relative cell state frequencies in mutant (right) and non-mutant (left) cells following GoT of sample N122 (DNMT3A R882 mutated, VAF=0.07). (2H) CH frequency (by gene) in age-and sex-matched high (red) and low (black) RDW individuals in a cohort of 18,147 individuals.

FIGS. 3A-3K: (3A) Analysis of age-linked compositional differences in MEBEMP (MEBEMP-E, MEBEMP-L, ERYP, BEMP, left) and CLP (CLP-E, CLP-M, CLP-L, right) populations, comparing specific cell state frequencies (out of total CD34+ population) in young (<50 years) vs. old (>60-years) individuals without clonal hematopoiesis, performed for males (blue) and females (red) separately. Kruskal-Wallis p values for group differences are denoted on top. (3B) Analysis of age-linked compositional differences within the MEBEMP differentiation trajectory, comparing the abundance of more (MEBEMP-L) to less (MPP) differentiated states in young (<50 years) vs. old (>60-years) individuals, performed for males (blue) and females (red) separately. The Kruskal-Wallis p value for difference among these groups is denoted on top. (3C) as in 3A, for the HSC population. (3D) Analysis of age-linked differences in (CD34+) cHSPC frequencies from total PBMCs in a recent cohort of 1000 healthy individuals undergoing PBMC scRNA-seq28. (3E) True age (x) vs age predicted based on composition-controlled MEBEMP expression (y). (3F) gene-gene correlation heatmap, calculated over individual-level HSC-MEBEMP gene expression controlled for HSC-MEBEMP composition. (3G) intra-individual correlation of LMNA signatures in CLPs and MEBEMPs, for both males (blue) and females (red). (3H) Analysis of age-linked differences in LMNA signature expression for CLP (right) and MEBEMP (left) populations in young (<50 years) vs. old (>60-years) individuals. Y axis denotes log2(observed/expected expression) normalized for composition. Boxplot centers, hinges and whiskers represent median, first and third quartiles and 1.5× interquartile range, respectively. (3I) individual heatmaps of single cell counts over 20 bins of stemness (AVP signature, y axis) and MEBEMP differentiation (GATA1 signature, x axis). Individual identifier, as well as his/her RBC, and MCV are denoted on top. (3J) comparison between individual sync scores and clinical parameters (RBC/MCV) across males. High and low sync scores (denoted by red and black dots respectively) define clinically distinct populations. (3K) correlation between individual sync scores and cell state compositions. Permutation test p value denoted on top.

FIGS. 4A-4G: (4A) composition bias score variation with age. (4B) cell type-specific comparison of S-phase signatures in circulating (left) vs. BM (right) HSPCs. (4C) S-phase signature variation with age in the late MEBEMP trajectory. (4D) corresponding individual S-phase signatures (X axis) and composition bias scores (Y) for individuals younger (left) and older (right) than 65 years. (4E-4G) like 4D, but showing the (4E) LMNA signature, (4F) sync scores, and (4G) RDW instead of S-phase, respectively.

FIGS. 5A-5F: (5A) diagnostic approach to leukemia analysis using the cHSPC reference atlas (scheme): 1. scRNA-seq on CD34-enriched PB and construction of a patient-specific metacell model, 2. Projection of patient derived metacells on the healthy reference atlas. 3. Compositional (relative cell state frequency) analysis and 4. Composition-controlled differential gene expression analysis. 5. Mutational and CNV analysis using targeted DNA sequencing and RNA-based karyotyping. (5B) projection of metacells derived from 2 new healthy individuals (not included in the reference model), 3 CMML patients, 2 MDS patients (1 of them prior to and following treatment), 1 MDS/MPN overlap patient, 1 MF patient and 2 AML patients on the healthy HSPC reference metacell model. Gene expression correlations between patient (projected) and reference metacells are color coded according to the legend on the right. (5C-5D) individual cell state frequency profiles over the HSC-MEBEMP and HSC-CLP differentiation gradients for 2 healthy and 7 patient samples (1 of them prior to and following treatment initiation). Dashed lines in 5D represent the median (black) and 5th and 95th percentiles (grey) of the healthy population. (5E) Compositional-controlled differential gene expression of 2 healthy and 7 patient samples (1 of them prior to and following treatment initiation) to the normal reference, quantifying the number of differentially expressed genes and identifying specific genes recurrently induced or repressed in disease. (5F) scRNA-seq karyotyping for 2 AML patients and 1 MDS patient prior to and following treatment initiation. Metacell models were created for each MDS/AML patient and projected over our healthy reference map. Coupled reference and projected (patient) metacells were then used for calculating expression ratios over all expressed genes in all chromosomes. Log2 fold-change expression (patient/healthy) for all expressed genes across all chromosomes is shown. Red lines represent the median of each chromosomal fold-change distribution.

FIG. 6: A block diagram, depicting a computing device which may be included in a system for determining a Hematopoietic Stem Cells (HSC) condition in a subject, according to some embodiments of the invention.

FIGS. 7A-7B: Block diagrams, depicting systems for determining (7A) and indication or (7B) an IPSS-M score in a subject according to some embodiments of the invention.

FIG. 8: A flow diagram, depicting a method of determining an HSC condition in a subject according to some embodiments of the invention.

FIG. 9: Factors involved in BEMP and NKTDP differentiation. Factors positively (CNRIP1, HPGDS, TET2, TNFSF10) and negatively (CD34, HBD, CD74 and BLVRB) regulated in the early stages of BEMP specification.

FIG. 10: True age (x) vs age predicted based on composition-controlled CLP expression (y).

FIG. 11: gene-gene correlation heatmap, calculated over individual-level CLP gene expression controlled for CLP composition.

FIGS. 12A-12C: (12A) heatmap of individual LMNA signature expression across the MEBEMP trajectory. Individual age and sex are color-coded on top. (12B) LMNA signature expression correlations between 39 technical & 20 biological replicates and their original samples. (12C) sync score correlations between 39 technical & 20 biological replicates and their original samples. All biological replicates were sampled 1 year following original blood draw.

FIGS. 13A-13C: (13A) each of 4 panels refers to a different cell state gene signature as noted on the x-axis. Panel top-boxplots of gene module expression distributions for different cell states in our reference atlas. Panel bottom-Gene signature expression density plots for each of the AML subclones. Reference gene signature distributions (panel top) were used to identify subpopulations of AML cells with CLP, MEBEMP, HSC and NKTDP characteristics (panel bottom). Dashed lines represent the threshold for expressing a gene signature, and the fraction of cells expressing a signature per AML clone is listed. (13B) left—correlation heatmap of differentially expressed gene signatures for AML-1 (N186, top) and AML-2 (N205, bottom). The malignant state is characterized by multiple novel gene signatures in addition to aberrant expression of “healthy” differentiation-related modules, right—UMAP projection of the metacell models of AML-1 (top) and AML-2 (bottom), colored by relative expression of differentially expressed genes. Overexpression of BCL2 in AML-1-2 compared to AML-1-1 can be seen. AML-1 gene signature: BCL2, VPREB1, RUNX3, GATA2, SELL, LMNA, ID2. AML-2 gene signature: CCL4, MPO, LMNA, MME, JCHAIN, ACY3, DNTT, GATA2. (13C) Expression heatmap of several genes across reference cell states and AML subclones. The malignant state differs greatly from the healthy state both in the expression of reference genes and by multiple additional gene expression signatures.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

The present invention, in some embodiments, provides non-invasive methods of detecting pathology of the bone marrow comprising receiving a subject cellular dataset based on single cell RNA sequencing (scRNA-seq) of CD34 positive cells from peripheral blood of the subject and analyzing the received subject cellular dataset in relation to a control dataset Non-invasive methods of predicting the percentage of blasts in the bone marrow comprising applying a trained machine learning model to a received subject cellular dataset based on single cell RNA sequencing (scRNA-seq) of CD34 positive cells from peripheral blood are also provided. Non-invasive methods of calculating an IPSS-M risk score are also provided. Systems for performing the methods of the invention are also provided.

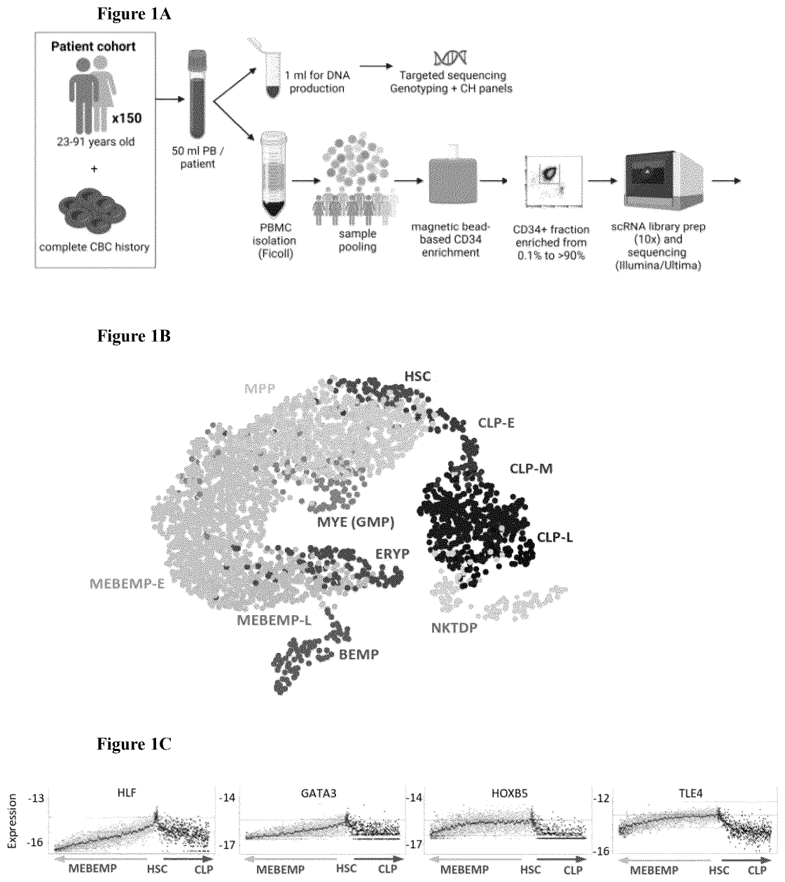

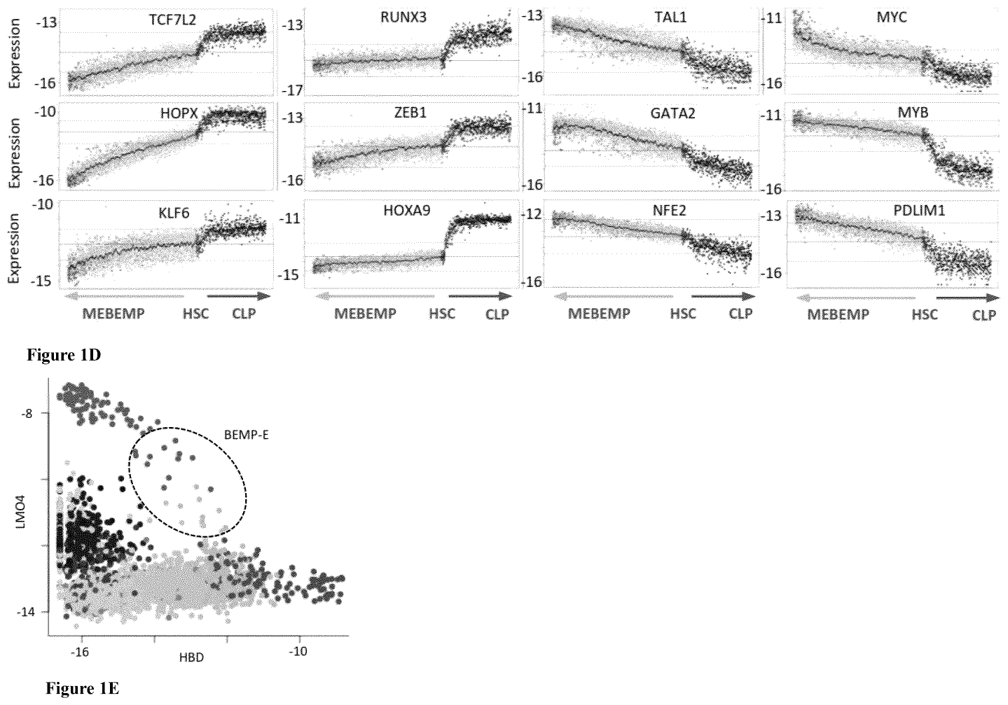

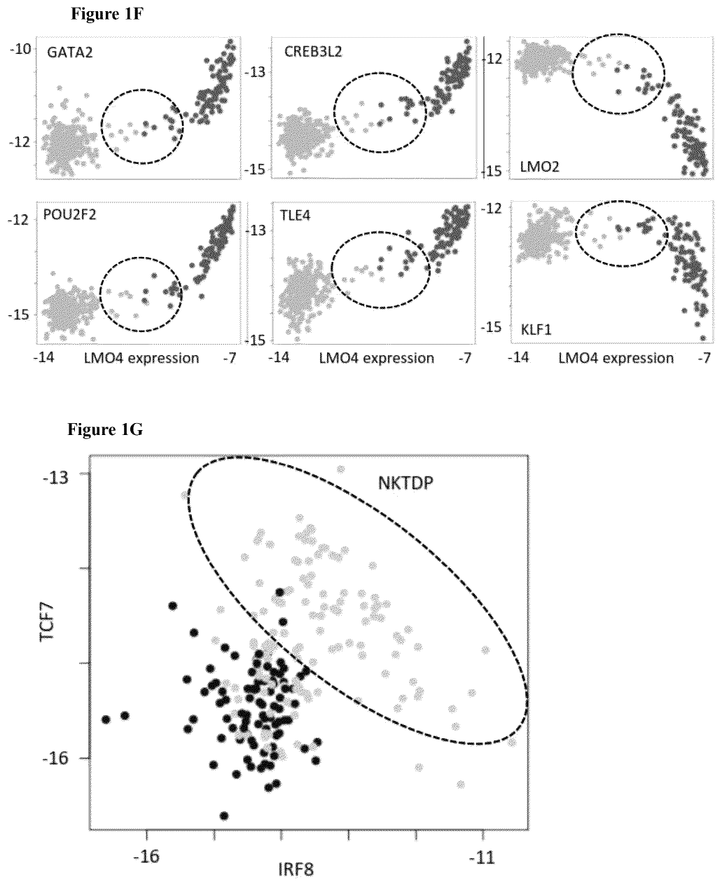

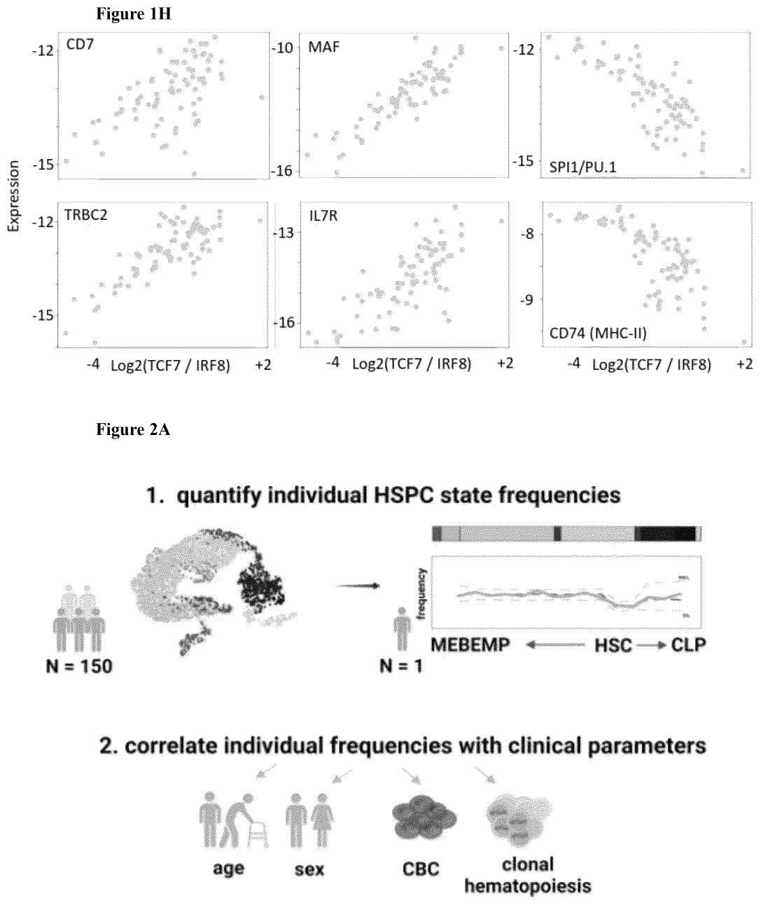

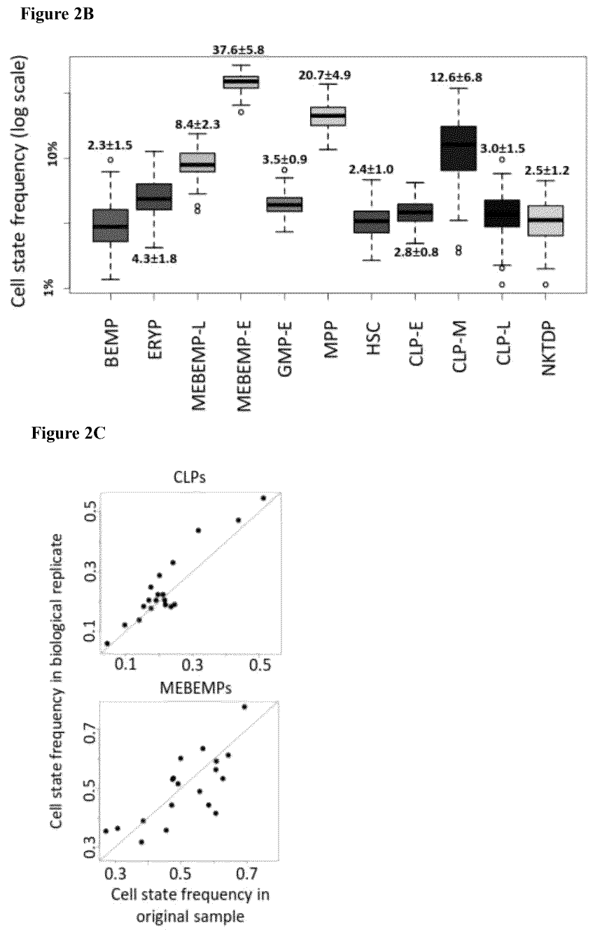

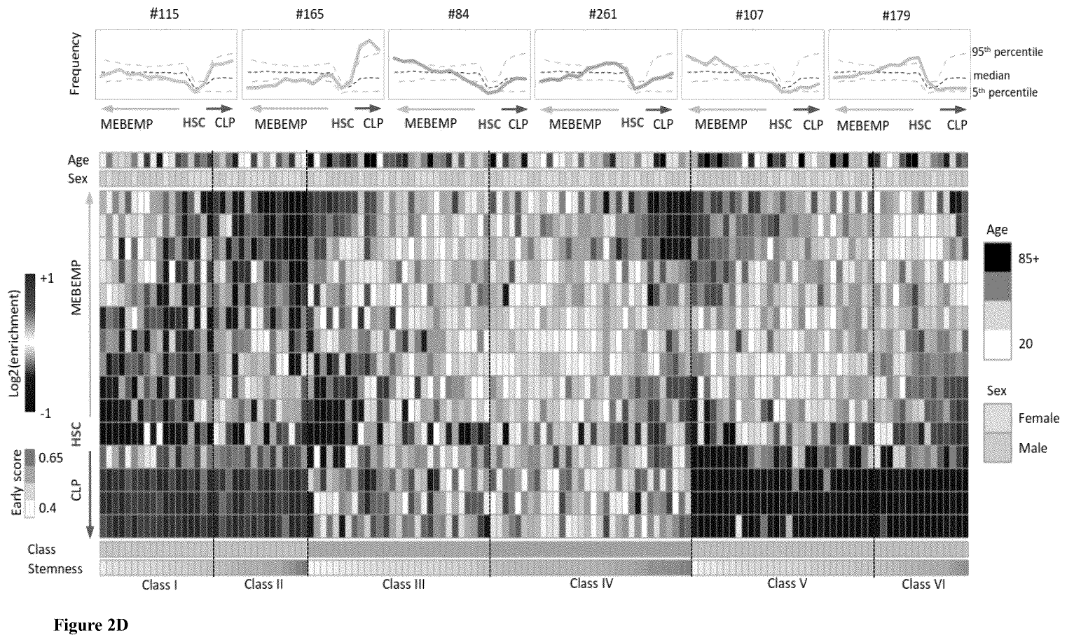

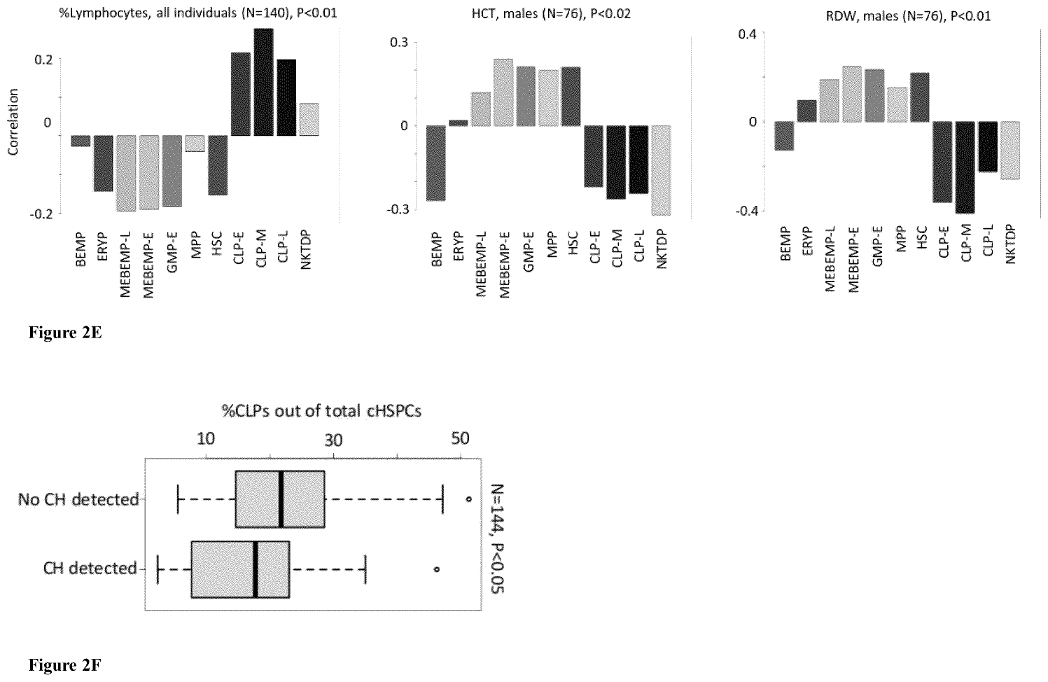

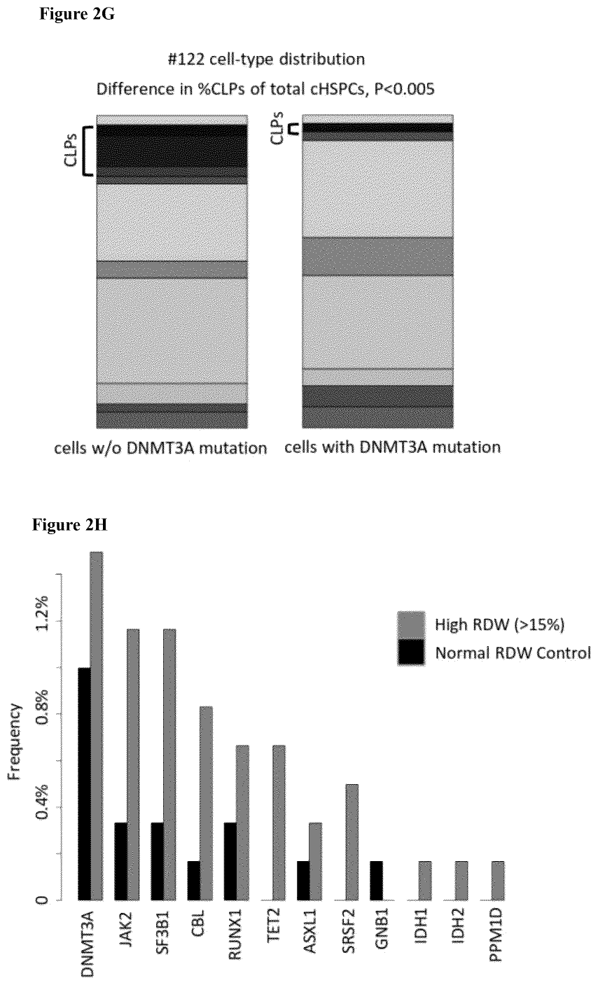

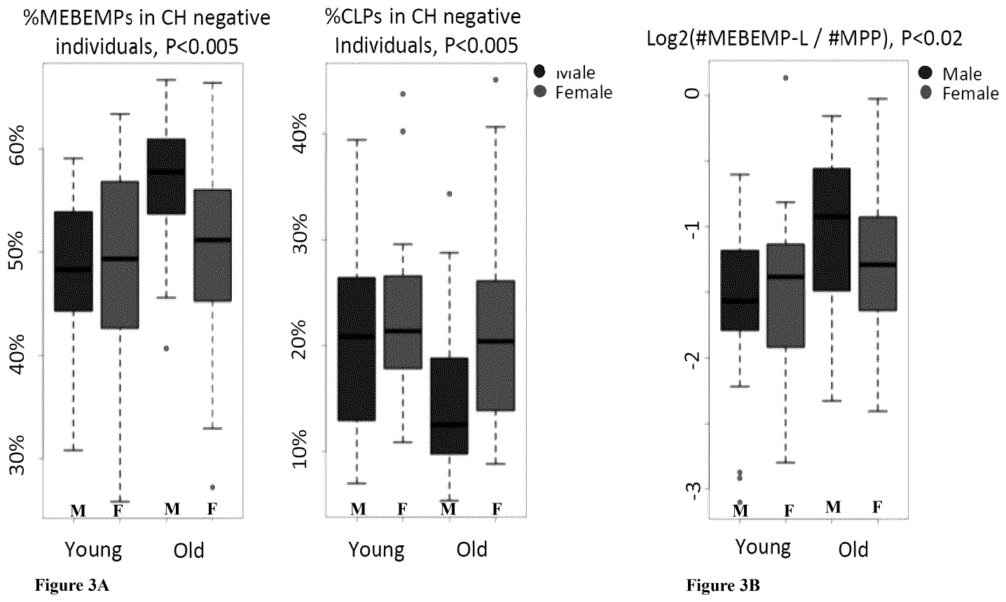

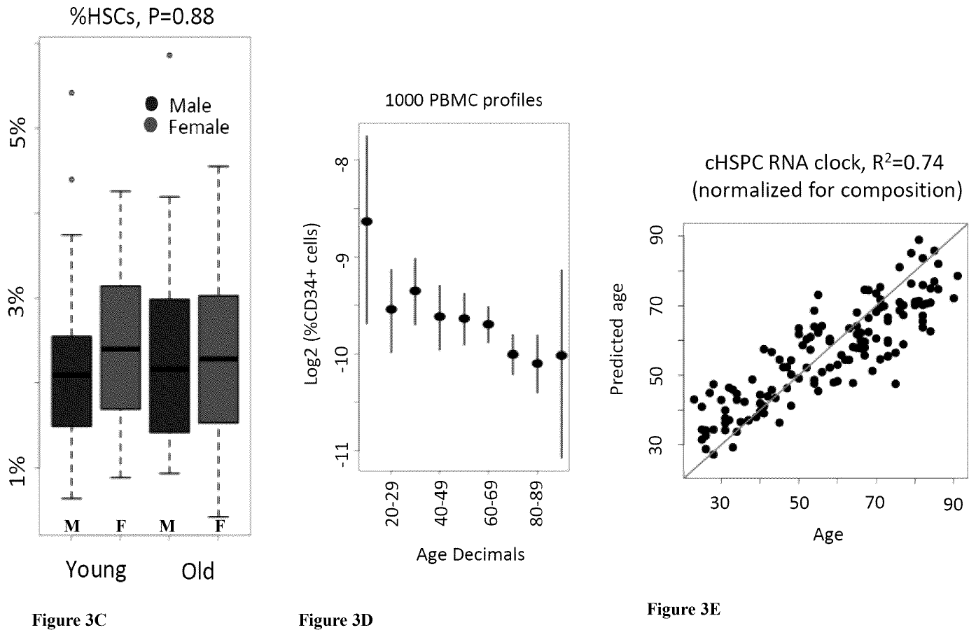

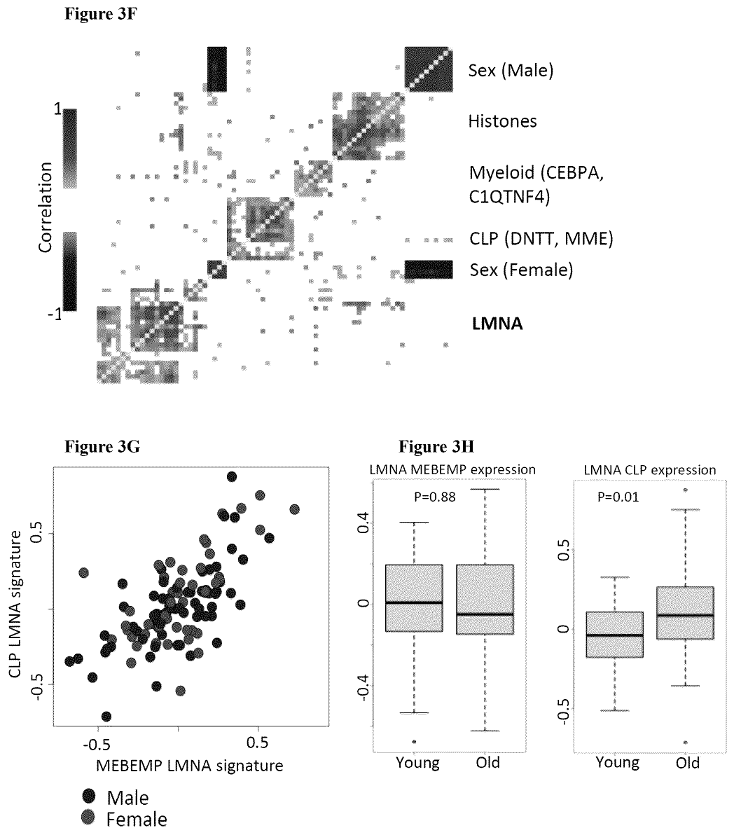

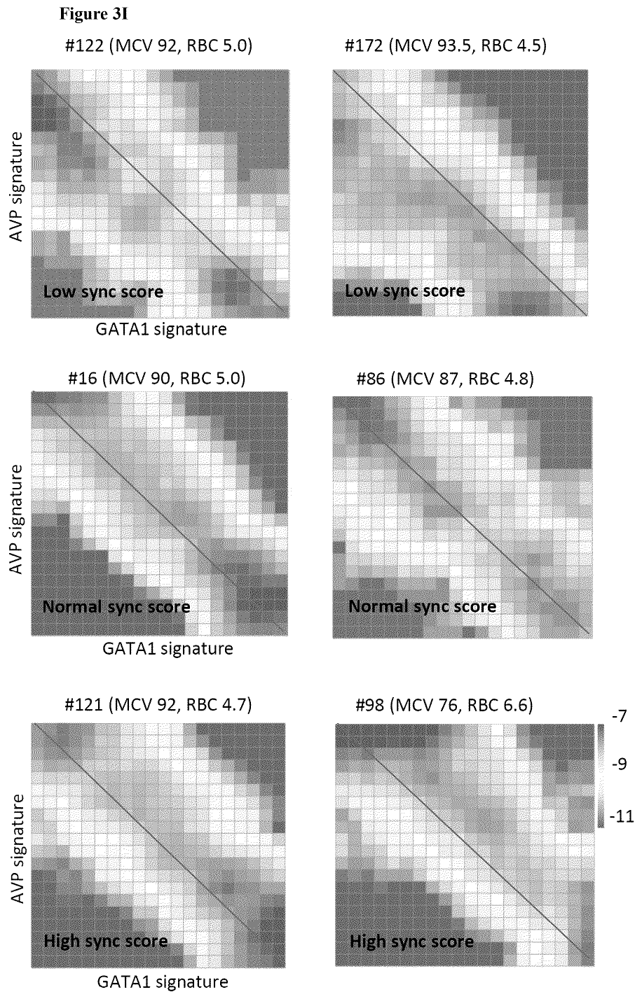

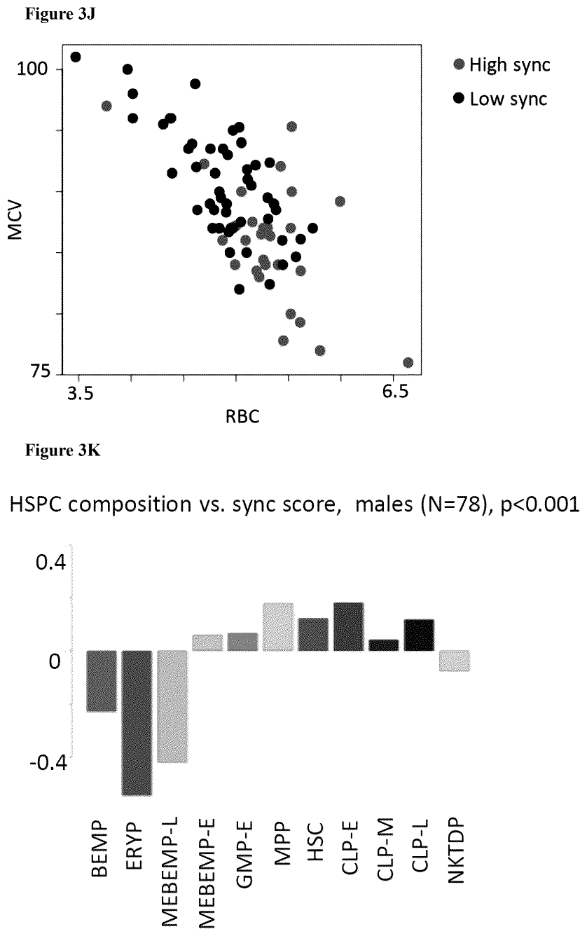

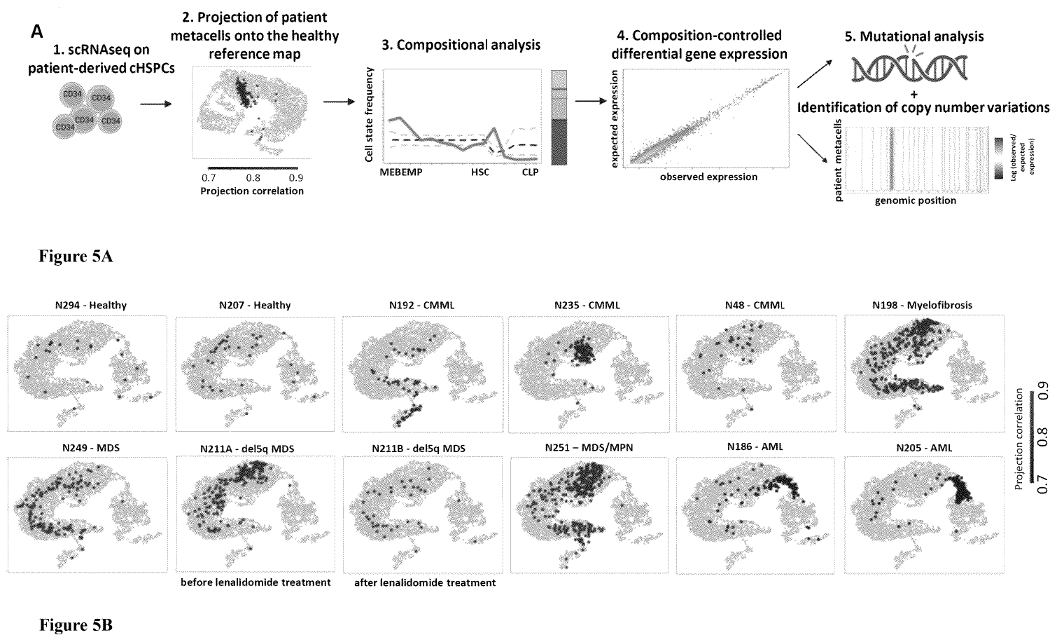

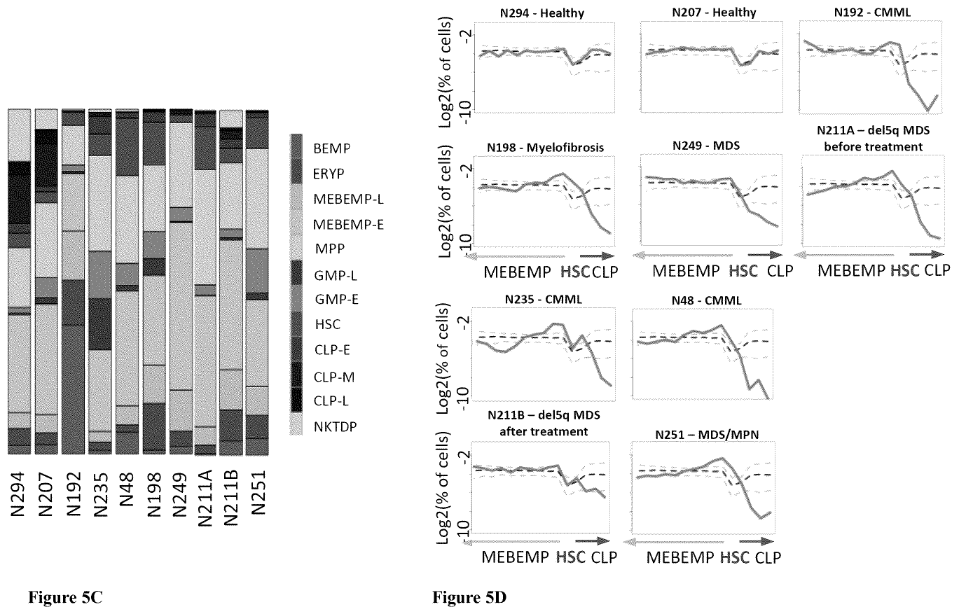

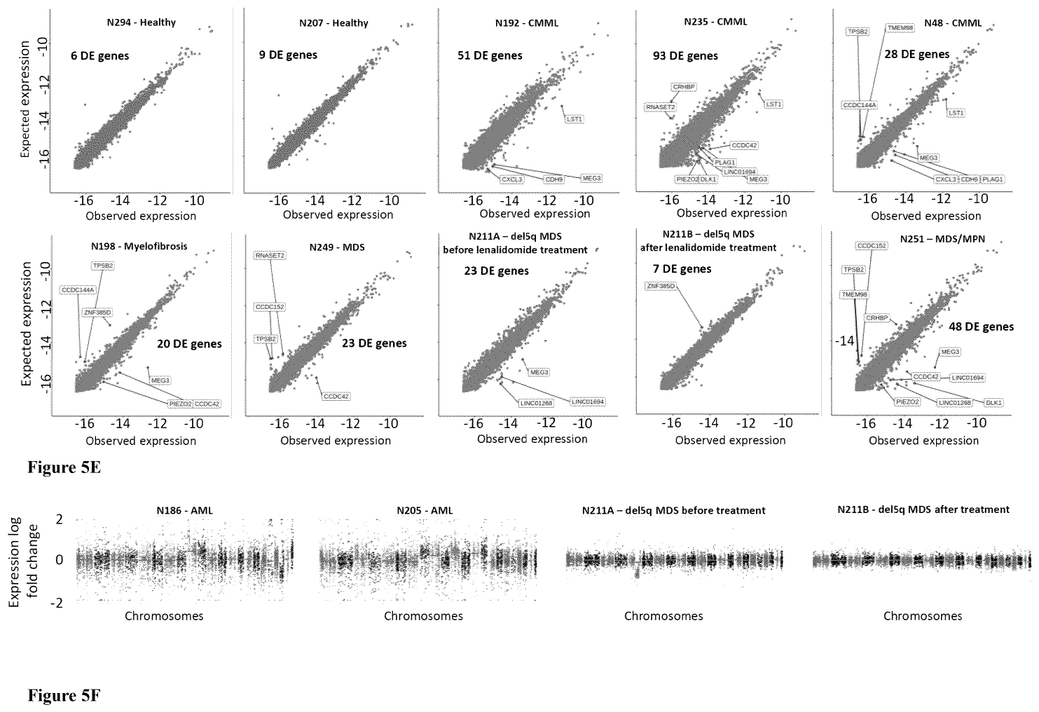

The present invention is based, at least in part, on the surprising finding that single cell RNA-sequencing (scRNA-Seq) of HSPCs in the blood can be used to recapitulate the status of HSPCs in the bone marrow and thereby detect bone marrow pathology, detect the presence and percentage of bone marrow blasts and predicts clinical outcome and treatment based on a divergence from what is observed in healthy controls. The current study characterizes inter-individual heterogeneity in cHSPCs across 148 healthy individuals, analyzing 627K PB CD34+ cells via scRNA-seq. The magnitude of the cohort, along with the potency and resolution of modern single cell technologies and the computational methods used herein, allowed the inventors to characterize in detail the transcriptional programs of diverse, sometimes rare (NKTDP, BEMP), HSPC sub-populations, refining and augmenting previous findings from much smaller cohorts (FIG. 1). The disclosure defines a normal reference range for cHSPC subpopulation frequencies within a large age-and sex-diverse healthy population and shows that cHSPC subtype compositions are highly variable between individuals, while the cell states themselves are remarkably universal (FIG. 2). These compositions remained stable over a one-year follow-up period, placing them as a strong individual characteristic. At the population level, a significant correlation between low CLP frequencies, CH and increased RDW was discovered (FIG. 2). The disclosure further shows that the known age-related myeloid bias in HSPCs is significantly male driven (FIG. 3). Analysis of composition-controlled transcriptional variance identified an RNA expression clock which correlated with age (FIG. 3), and a novel gene module (LMNA) whose expression increased in CLPs with age. It was discovered that healthy individuals regulate the transition from stemness to myeloid differentiation differently and found that premature down-regulation of stemness genes correlates with high MCV anemia (FIG. 3). Finally, it was demonstrating how this resource can be used to effectively identify and diagnose pathological cases based on their abnormal cHSPC subpopulation frequencies, differential gene signatures, and chromosomal aberrations (FIG. 5), all from peripheral blood samples. A detailed pipeline for the identification and characterization of such pathologies based on the normal reference is provided.

By a first aspect, there is provided a method of analyzing the bone marrow of a subject, the method comprising:
a. receiving a dataset based on CD34 positive cells from blood of the subject; and
b. analyzing the received subject dataset in relation to a control dataset,
   thereby analyzing bone marrow of a subject

In some embodiments, the method is a diagnostic method. In some embodiments, the method is a prognostic method. In some embodiments, the method is a non-invasive method. In some embodiments, the method is an in vitro method. In some embodiments, the method is an ex vivo method. In some embodiments, the method is a method of treatment. In some embodiments, the method is a computerized method. In some embodiments, the method is performed by at least one processor. In some embodiments, the method requires analyzing data that is beyond the capability of the human mind.

As used herein, the term “non-invasive” refers to a method that does not require extraction of a sample from the bone marrow. Bone marrow biopsies and aspirations are invasive, painful and expensive procedures that provide a diagnostician with a sample of cells in the bone marrow. The instant method circumvents the drawbacks of invasive bone marrow samples by analyzing the bone marrow via the circulating CD34 positive cells found in blood. Thus, the instant method is highly beneficial as it is non-invasive. In some embodiments, blood is peripheral blood. In some embodiments, blood is venous blood. In some embodiments, blood is circulating blood. In some embodiments, blood is not from an organ. In some embodiments, blood is not from tissue. In some embodiments, blood is not from the bone marrow. In some embodiments, blood is a blood sample.

In some embodiments, the CD34 positive cells are hematopoietic stem progenitor cells (HSPCs). CD34 is a transmembrane cell surface protein that marks hematopoietic stem cells (HSCs) as well as early progenitor cells that have differentiated from HSCs. CD34positive cells run the gamut from fully stem cells (HSCs) to cells that have begun to differentiate toward one of two lineage programs: common lymphoid progenitor (CLP) lineage or megakaryocyte/erythrocyte/basophil/eosinophil/mast progenitors (MEBEM-P) lineage. The human CD34 protein sequence can be found in Uniprot entry P28906 while the Entrez gene ID is #947. Agents that bind to and/or identify CD34 expressing cells are well known in the art, as are kits for isolation of CD34 positive cells. Examples include but are not limited to Dynabead CD34 Positive Isolation Kit (ThermoFisher), I-O Human CD34+ Cell Isolation Kit (Creative Biolabs), EasySep Human CD34 Positive Selection Kit (Stemcell Technologies) and CD34 MicroBead Kit, human (Miltenyi Biotec).

In some embodiments, the dataset is based on CD34 positive cells from a blood sample from the subject. In some embodiments, the dataset is based on all CD34 positive cells in the sample. In some embodiments, the dataset is a cellular dataset. In some embodiments, the dataset is an ensemble of the CD34 positive cells in the blood. In some embodiments, the dataset is a per cell dataset. In some embodiments, the dataset contains an entry for each CD34 positive cell. In some embodiments, the data is data on the totality of CD34 positive cells in the blood. In some embodiments, the dataset is statistical data. In some embodiments, statistical data is statistical data is a data transformation of the cellular data. In some embodiments, the dataset is based on single cell data. In some embodiments, the dataset comprises single cell data. In some embodiments, the dataset consists of single cell data. In some embodiments, the single cell data is single cell RNA data. In some embodiments, the single cell RNA data is single cell RNA sequencing (scRNA-seq) data. In some embodiments, the data is reads. In some embodiments, reads are sequencing reads. In some embodiments, the data is transcriptome data. In some embodiments, the single cell data is protein data. In some embodiments, the single cell data is proteome data. In some embodiments, the dataset comprises a transcriptome of each of the CD34 positive cells. In some embodiments, the dataset comprises the proteome of each of the CD34 positive cells. In some embodiments, the dataset is a cell atlas. In some embodiments, the cell atlas is annotated. In some embodiments, the annotation is the cell type.

In some embodiments, the method further comprises receiving a blood sample from the subject. In some embodiments, the method further comprises extracting a blood sample from the subject. In some embodiments, a blood sample is a peripheral blood sample. In some embodiments, the method further comprises producing a dataset from the sample. In some embodiments, the method further comprises isolating CD34 positive cells from the sample. In some embodiments, isolating comprises extracting. In some embodiments, isolating is positive selection. In some embodiments, isolating is negative selection.

In some embodiments, the method comprises sequencing the CD34 positive cells. In some embodiments, sequencing is single cell sequencing. In some embodiments, the sequencing is next generation sequencing. In some embodiments, the sequencing is high throughput sequencing. In some embodiments, the sequencing is massively parallel sequencing. In some embodiments, the dataset is a dataset of sequences. In some embodiments, the dataset is a dataset of expression. In some embodiments, expression is gene expression.

In some embodiments, CD34 cells are clustered into cell types. In some embodiments, cell types are defined by their transcriptional profile. In some embodiments, cell types are defined by their transcriptome. In some embodiments, cell types are defined by their proteome. In some embodiments, cell types are defined by their level of differentiation. In some embodiments, cell types are defined by their differentiation status. In some embodiments, cell types are defined by how similarly they have differentiated.

In some embodiments, the dataset is a metacell model of the CD34 positive cells. In some embodiments, the model is of the totality of CD34 positive cells. Metacell modeling computes partitions of cells by similarity to produce mostly homogenous groups (e.g., cell types) which are defined as metacells. In some embodiments, a cell type comprises a plurality of metacells. In some embodiments, the cell type comprises metacells with similar differentiation. Methods of producing metacells from single cell data are well known and are described hereinbelow as well as for example in Baran, et al., “MetaCell: analysis of single-cell RNA-seq data using K-nn graph partitions”, Genome Biol. 2019 Oct. 11;20(1):206 and Ben-Kiki et al., “Metacell-s: a divide and conquer metacell algorithm for scalable scRNA-seq analysis”, Genome Biol. 2022 Apr. 19;23(1):100 the contents of which are hereby incorporated herein by reference in their entirety. Further, the metacell program is freely available at github. com/tanaylab/metacells. In some embodiments, the method comprises generating metacells from the scRNA-seq data.

In some embodiments, the control dataset comprises the same type of data as the subject dataset. In some embodiments, the control dataset comprises a plurality of subject datasets. In some embodiments, the control dataset comprises a plurality of datasets. In some embodiments, each of the plurality of datasets in the control dataset is from a different control subject. In some embodiments, the control dataset comprises a plurality of control subject datasets. In some embodiments, each dataset of the plurality is based on scRNA-seq of CD34 positive cells. In some embodiments, the CD34 positive cells are from blood. In some embodiments, the CD34 positive cells are from control subjects. In some embodiments, control subjects are healthy subjects. In some embodiments, control subjects are subjects with a pathology of the bone marrow. In some embodiments, control subjects are both healthy subjects and subjects with a pathology of the bone marrow. In some embodiments, the control dataset is an atlas of control cells. In some embodiments, the control dataset is an atlas of metacells from control subjects. In some embodiments, the atlas is an atlas of datasets.

In some embodiments, a dataset comprises grouping of the cells into cell types. In some embodiments, the metacells are grouped into cell types. In some embodiments, cell types share a common transcription profile. In some embodiments, cell types share a common differentiation state. In some embodiments, the differentiation state is within the HSPC spectrum of differentiation. In some embodiments, the control dataset comprises amounts of cell types in control subjects. In some embodiments, amounts are ranges. In some embodiments, cell types are types of metacells. In some embodiments, cell types are differentiation states. In some embodiments, amounts are relative amounts. In some embodiments, amounts are amounts of all cell types in a control subject. In some embodiments, ranges are ranges of all cell types in control subjects.

In some embodiments, the cell types are selected from different differentiation states of the CD34 positive cells. In some embodiments, the cell types are selected from hematopoietic stem cells (HSC), common lymphoid progenitor cells (CLP), natural killer/T/dendritic cell progenitor cells (NKTDP), multipotent progenitor cells (MPP), early granulocyte-monocyte progenitor cells (GMP-E), megakaryocyte/erythrocyte/basophil/eosinophil/mast progenitor cells (MEBEMP), erythrocyte progenitor cells (ERYP) and basophil/eosinophil/mast progenitor cells (BEMP). In some embodiments, CLPs comprise early CLPs (CLP-E), intermediate CLPs (CLP-M) and late CLPs (CLP-L). In some embodiments, MEBEMPs comprise early MEBEMPs (MEBEMP-E) and late MEBEMPs (MEBEMP-L). In some embodiments, the cell types are selected from BEMP, ERYP, MEBEMP-L, MEBEMP-E, GMP-E, MPP, HSC, CLP-E, CLP-M, CLP-L and NKTDP. In some embodiments, CLP comprises NKTDP. In some embodiments, CLP comprises CLP-E, CLP-M, CLP-L and NKTDP. In some embodiments, MEBEMP comprises BEMP. In some embodiments, MEBEMP comprises ERYP. In some embodiments, MEBEMP comprises BEMP, ERYP and MEBEMP-L.

In some embodiments, the control dataset comprises control ranges for each cell type. In some embodiments, control ranges are relative ranges. In some embodiments, relative ranges are relative abundance. In some embodiments, control relative ranges are relative percentage of all CD34 positive cells. In some embodiments, percentage is percent of CD34 positive cells in a sample. In some embodiments, the control ranges are provided in FIG. 2B. In some embodiments, the control range for BEMP is about 4.4% of CD34 positive cells. In some embodiments, about 4.4% is 4.4+/−4.1%. In some embodiments, the control range for ERYP is about 1.4% of CD34 positive cells. In some embodiments, about 1.4% is 1.4+/−0.7%. In some embodiments, the control range for MEMBEMP-L is about 8.2% of CD34 positive cells. In some embodiments, about 8.2% is 8.2+/−2.2%. In some embodiments, the control range for MEMBEMP-E is about 38.0% of CD34 positive cells. In some embodiments, about 38.0% is 38.0+/−6.5%. In some embodiments, the control range for GMP-E is about 3.0% of CD34 positive cells. In some embodiments, about 3.0% is 3.0+/−0.9%. In some embodiments, the control range for MPP is about 21.6% of CD34 positive cells. In some embodiments, about 31.6% is 31.6+/−4.7%. In some embodiments, the control range for HSC is about 1.8% of CD34 positive cells. In some embodiments, about 1.8% is 1.8+/−1.1%. In some embodiments, the control range for CLP-E is about 2.5% of CD34 positive cells. In some embodiments, about 2.5% is 2.5+/−0.8%. In some embodiments, the control range for CLP-M is about 7.9% of CD34 positive cells. In some embodiments, about 7.9% is 7.9+/−5.2%. In some embodiments, the control range for CLP-L is about 5.7% of CD34 positive cells. In some embodiments, about 5.7% is 5.7+/−3.6%. In some embodiments, the control range for NKTDP is about 5.1% of CD34 positive cells. In some embodiments, about 45.1% is 5.1+/−3.0%.

In some embodiments, analyzing is comparing. In some embodiments, analyzing comprises projecting the dataset onto the control dataset. In some embodiments, the analyzing is determining cell type differences between the subject dataset and the control dataset. In some embodiments, changes are loss of cells of a cell type. In some embodiments, changes are gains of cells of a cell type. In some embodiments, cells are metacells. In some embodiments, analyzing is analyzing the totality of the subject dataset. In some embodiments, analyzing is analyzing the subject dataset in relation to all of the plurality of datasets within the control dataset.

In some embodiments, analyzing bone marrow comprises detecting a pathology of the bone marrow. In some embodiments, detecting comprises determining the pathology of the bone marrow. In some embodiments, analyzing comprises diagnosing a pathology of the bone marrow. In some embodiments, analyzing comprises prognosing a pathology of the bone marrow. In some embodiments, analyzing comprises determining the proper treatment of a pathology of the bone marrow. In some embodiments, analyzing comprises determining the amount of blasts in the bone marrow. In some embodiments, determining is predicting. In some embodiments, determining is estimating. In some embodiments, determining is approximating. In some embodiments, the determining is without actually counting blasts in the bone marrow.

In some embodiments, deviation of the subject dataset from the control dataset indicates a bone marrow pathology. In some embodiments, deviation of the subject dataset from the control dataset indicates a specific bone marrow pathology. In some embodiments, deviation of the subject dataset from the control dataset indicates a disease of the bone marrow. In some embodiments, deviation comprises a difference when the subject dataset is projected onto the control dataset. In some embodiments, deviation is higher levels/amounts of a cell type being present in the subject than the healthy controls. In some embodiments, deviation is a higher frequency of a cell type in the subject than the healthy controls. In some embodiments, deviation is lower levels/amounts of a cell types being present in the subject than the healthy controls. In some embodiments, deviation is a lower frequency of a cell type in the subject than the healthy controls. In some embodiments, lower amounts is the absence of a cell type. In some embodiments, higher amounts is the presence of new cell type.

As used herein, the term “pathology of the bone marrow” refers to any disease or condition affecting the bone marrow of humans. In some embodiments, a pathology is a disease. In some embodiments, a pathology is an abnormality of the bone marrow. Examples of bone marrow pathologies include but are not limited to: myelodysplastic syndrome (MDS), Chronic myelomonocytic leukemia (CMML), Chronic myeloid leukemia (CML), Acute myeloid leukemia (AML), polycythemia vera (PV), essential thrombocythemia (ET), Mastocytosis, chronic eosinophilic leukemia, primary myelofibrosis (MF), post-ET myelofibrosis, post PV myelofibrosis, acute lymphoblastic leukemia (ALL), acute leukemia of ambiguous lineage, multiple myeloma (MM), myeloproliferative neoplasm (MPN) and blastic plasmacytoid dendritic cell leukemia. In some embodiments, the pathology is cancer. In some embodiments, the cancer is a hematopoietic cancer. In some embodiments, the cancer is leukemia. In some embodiments, the pathology is MDS. MDS is a well-known group of cancers in which immature blood cells (HSPCs) within the bone marrow do not mature to become healthy blood cells. In some embodiments, the pathology is CMML. In some embodiments, the pathology is MF. In some embodiments, MF is selected from primary MF, post-ET MF and post PV MF. In some embodiments, the pathology is MPN. In some embodiments, the pathology is MDS/MPN. In some embodiments, the pathology is AML. In some embodiments, the pathology is not AML. In some embodiments, the pathology is selected from the group consisting of: MDS, CMML, MF, and MPN. In some embodiments, the pathology is selected from the group consisting of: MDS, CMML, MF, and MDS/MPN. In some embodiments, the pathology is selected from the group consisting of: MDS, CMML, MF, MPN and AML. In some embodiments, the pathology is selected from the group consisting of: MDS, CMML, MF, MDS/MPN and AML. In some embodiments, MDS is MDS with a del5q mutation.

In some embodiments, the method is a method of detecting MDS. In some embodiments, deviation in the amount or frequency of ERYP cells indicates the presence of MDS. In some embodiments, deviation in the amount or frequency of BEMP cells indicates the presence of MDS. In some embodiments, deviation in the amount or frequency of MEBEMP cells indicates the presence of MDS. In some embodiments, deviation in the amount or frequency of any one of ERYP, BEMP and MEBEMP cells indicates the presence of MDS. In some embodiments, deviation in the amount or frequency of all of ERYP, BEMP and MEBEMP cells indicates the presence of MDS. In some embodiments, MEBEMP is MEBEMP-L or MEBEMP-E. In some embodiments, MEBEMP is MEBEMP-L and MEBEMP-E. In some embodiments, the deviation is an increase. In some embodiments, deviation in the amount or frequency of CLP cells indicates the presence of MDS. In some embodiments, the deviation is a decrease. In some embodiments, CLP is CLP-L, CLP-M or CLP-E. In some embodiments, CLP is any two of CLP-L, CLP-M and CLP-E. In some embodiments, CLP is CLP-L, CLP-M and CLP-E. In some embodiments, decrease in CLP amount or frequence indicates the presence of MDS. In some embodiments, MDS is MDS/MPN. In some embodiments, decrease in CLP amount or frequence indicates the presence of MDS or MPN. In some embodiments, deviation in the amount or frequency of NKTDP cells indicates the presence of MDS. In some embodiments, decrease in NKTDP amount or frequence indicates the presence of MDS. In some embodiments, MDS is MDS/MPN. In some embodiments, decrease in NKTDP amount or frequence indicates the presence of MDS or MPN.

In some embodiments, the method is a method of detecting CMML. In some embodiments, deviation in the amount or frequency of GMP cells indicates the presence of CMML. In some embodiments, GMP is GMP-E. In some embodiments, the deviation is an increase. In some embodiments, deviation in the amount or frequency of CLP cells indicates the presence of CMML. In some embodiments, the deviation is a decrease. In some embodiments, CLP is CLP-L, CLP-M or CLP-E. In some embodiments, CLP is any two of CLP-L, CLP-M and CLP-E. In some embodiments, CLP is CLP-L, CLP-M and CLP-E.

In some embodiments, the method is a method of detecting MF. In some embodiments, deviation in the amount or frequency of CLP cells indicates the presence of MF. In some embodiments, the deviation is a decrease. In some embodiments, deviation in the amount or frequency of NKTDP cells indicates the presence of MF. In some embodiments, decrease in NKTDP amount or frequence indicates the presence of MF.

In some embodiments, the method is a method of detecting MPN. In some embodiments, deviation in the amount or frequency of CLP cells indicates the presence of MPN. In some embodiments, the deviation is a decrease. In some embodiments, CLP is CLP-L, CLP-M or CLP-E. In some embodiments, CLP is any two of CLP-L, CLP-M and CLP-E. In some embodiments, CLP is CLP-L, CLP-M and CLP-E. In some embodiments, decrease in CLP amount or frequence indicates the presence of MPN. In some embodiments, MPN is MDS/MPN. In some embodiments, decrease in CLP amount or frequence indicates the presence of MDS or MPN. In some embodiments, deviation in the amount or frequency of NKTDP cells indicates the presence of MPN. In some embodiments, decrease in NKTDP amount or frequence indicates the presence of MPN. In some embodiments, MPN is MDS/MPN. In some embodiments, decrease in NKTDP amount or frequence indicates the presence of MDS or MPN.

In some embodiments, the method is a method of detecting AML. In some embodiments, deviation in the amount or frequency of CLP cells indicates the presence of AML. In some embodiments, CLP is CLP-L, CLP-M or CLP-E. In some embodiments, CLP is any two of CLP-L, CLP-M and CLP-E. In some embodiments, CLP is CLP-L, CLP-M and CLP-E. In some embodiments, deviation in the amount or frequency of NKTDP cells indicates the presence of AML. In some embodiments, the deviation is an increase. In some embodiments, an increase in the amount or frequency of NKTDP cells indicates the presence of AML.

In some embodiments, the pathology of the bone marrow comprises an increased percentage of blasts. In some embodiments, the pathology of the bone marrow is characterized by an increased percentage of blasts. In some embodiments, the pathology of the bone marrow is selected from AML and MDS. In some embodiments, MDS is MDS/MPN. In some embodiments, the pathology of the bone marrow is selected from AML, MPN and MDS. In some embodiments, AML and MDS are characterized by an increased percentage of blasts. In some embodiments, a deviation in the frequency of CLP-E indicates the presence of an increased amount of blasts. In some embodiments, a deviation is an increase. In some embodiments, an increase in CLP-E is the deviation. In some embodiments, the magnitude of the increase is proportionate to the increase in the amount of blasts. In some embodiments, an increase in blasts is as compared to the amount of blasts in a healthy control. In some embodiments, a healthy control is a healthy cohort. In some embodiments, the healthy cohort is the subjects that make up the control dataset. In some embodiments, a linear regression predicts the amount of blasts from the amount of CLP-E.

In some embodiments, analyzing comprises producing a feature vector representing deviation of the subject's cellular data from the control cellular data. In some embodiments, the feature vector comprises a plurality of entries. In some embodiments, each entry corresponds to a specific cell type. In some embodiments, each entry corresponds to an amount of each cell type. In some embodiments, the amount is the number. In some embodiments, the amount is the frequency. In some embodiments, the frequency is the percentage of all CD34 positive cells. In some embodiments, each entry represents or corresponds to the deviation from a reference value. In some embodiments, the deviation is the magnitude of deviation. In some embodiments, the reference value is the values from the control dataset. In some embodiments, the reference value is a range of the amount of a cell type. In some embodiments, a cell type is a cell population. In some embodiments, the range is the control range. In some embodiments, the range is the healthy range.

In some embodiments, analyzing comprises applying a trained machine learning model to the received dataset. In some embodiments, the machine learning model is trained on a training set. In some embodiments, the training set comprises the control dataset. In some embodiments, the training set comprises the plurality of cellular datasets. In some embodiments, the machine learning model outputs a classification of the subject's bone marrow. In some embodiments, the machine learning model outputs a classification of the subject. In some embodiments, the machine learning model outputs an analysis of the subject's bone marrow. In some embodiments, the classification is healthy or not.

In some embodiments, the training set comprises datasets from healthy subjects. In some embodiments, training set comprises datasets from subjects suffering from pathology of the bone marrow. In some embodiments, the training set comprises datasets from subjects suffering from a plurality of pathologies of the bone marrow. In some embodiments, the training set further comprises labels. In some embodiments, the labels label the datasets. In some embodiments, the labels indicate if the dataset is from a healthy subject or subject with a pathology of the bone marrow. In some embodiments, the label indicates the pathology of the bone marrow. In some embodiments, the label indicates the type of pathology. In some embodiments, classification is healthy or suffering from a pathology of the more marrow. In some embodiments, classification comprises classifying what the pathology is. In some embodiments, classification comprises classifying the type of pathology of the bone marrow.

In some embodiments, analyzing comprises applying a trained machine learning model to a parameter extracted from the dataset. In some embodiments, analyzing comprises applying a trained machine learning model to the feature vector. In some embodiments, the feature vector is a vector of the amounts of cell types. In some embodiments, cell types are all cell types of the CD34 positive cells in a sample. In some embodiments, the cell types are the full ensemble of CD34 positive cells in a sample. In some embodiments, the machine learning model is trained on a training set. In some embodiments, the training set comprises feature vectors from healthy subject. In some embodiments, the training set comprises parameters extracted from datasets from healthy subjects. In some embodiments, the training set comprises feature vectors from subject suffering from a bone marrow pathology. In some embodiments, the training set comprises parameters extracted from datasets from subjects suffering from a bone marrow pathology. In some embodiments, the training set comprises labels. In some embodiments, the labels indicate a feature vector is from a healthy subject or subject with a bone marrow pathology. In some embodiments, the labels indicate an extracted parameter is from a healthy subject or subject with a bone marrow pathology.

In some embodiments, analyzing further comprises applying a trained machine learning model to at least one clinical parameter. In some embodiments, the clinical parameter is a clinical parameter of the subject. In some embodiments, the clinical parameter is age. In some embodiments, the clinical parameter is sex. In some embodiments, the clinical parameter is sex and age. In some embodiments, the machine learning model is trained on a training set comprises at least one clinical parameter.

By another aspect, there is provided a method of predicting the amount of blasts in the bone marrow of a subject, the method comprising receiving a measure of the CLP-E cells in peripheral blood from the subject, thereby predicting the amount of blasts in the bone marrow of a subject.

In some embodiments, the measure of CLP-E cells is proportional to the amount of blasts in the bone marrow of the subject. In some embodiments, proportional is linearly proportional. In some embodiments, a linear regression indicates the amount of blasts from the measure of CLP-E. In some embodiments, indicates is predicts. In some embodiments, a measure above a predetermined threshold indicates blasts above a predetermined threshold. In some embodiments, the measure of CLP-E cells is the amount of CLP-E cells. In some embodiments, the measure of CLP-E cells is the number of CLP-E cells. In some embodiments, the measure of CLP-E cells is the proportion of CLP-E cells in the CD34 positive cells in the peripheral blood. CLP-E cells can be measured by any method known in the art, comprising flow cytometry, immunostaining, sequencing, producing of metacells from scRNA-seq and the like. Methods of identifying these cells in a sample, including a blood sample, are known in the art and any such method may be used. Methods of identifying CLP-E cells for example, are provided hereinbelow and in Ding and Morrison, “Haematopoietic stem cells and early lymphoid progenitors occupy distinct bone marrow niches”, Nature. 2013, Mar. 14; 495(7440): 231-235, the contents of which are herein incorporated by reference in their entirety.

In some embodiments, the method further comprises receiving a peripheral blood sample. In some embodiments, the method further comprises measuring CLP-E cells in the sample. In some embodiments, measuring is counting. In some embodiments, the method further comprises receiving scRNA-seq data from CD34 positive cells in the blood and calculating the number/amount/percentage of CLP-E cells in the blood. In some embodiments, in the blood is in the sample. In some embodiments, the method further comprises analyzing the received measure in relation to a control dataset.

By another aspect, there is provided a method of predicting the amount of blasts in the bone marrow of a subject, the method comprising:
a. receiving a dataset based on CD34 positive cells from blood of the subject; and
b. applying a trained machine learning model to the received dataset, wherein the machine learning model outputs a predicted amount of blasts in the bone marrow of the subject;
   thereby predicting the amount of blasts in the bone marrow of a subject.

In some embodiments, the subject is a mammal. In some embodiments, the mammal is a human. In some embodiments, the subject is in need of a method of the invention. In some embodiments, the subject is male. In some embodiments, the subject is female. In some embodiments, the subject suffers from a pathology of the bone marrow. In some embodiments, a bone marrow pathology is a bone marrow malignancy. In some embodiments, the subject suffers from leukemia. In some embodiments, leukemia is selected from AML, CMML, CML, Mastocytosis, chronic eosinophilic leukemia, acute leukemia of ambiguous lineage and blastic plasmacytoid dendritic cell leukemia.

In some embodiments, the amount of blasts is the number of blasts. In some embodiments, the amount of blasts is the frequency of blasts. In some embodiments, the amount of blasts is the percentage of blasts in the bone marrow. In some embodiments, percentage is relative to all cells in the bone marrow. In some embodiments, all cells are all CD34 positive cells.

In some embodiments, the training set comprises subjects suffering from MDS. In some embodiments, the training set comprises non-MDS subjects. In some embodiments, the training set comprises leukemic subject. In some embodiments, the training set comprises leukemic and non-leukemic subjects. In some embodiments, the training set further comprises labels. In some embodiments, the labels label the datasets. In some embodiments, the labels indicate the amount of blasts in the subject that provided the dataset. In some embodiments, the percentage of blasts in the bone marrow is known for each subject of the control dataset. In some embodiments, a subject of the control dataset is a subject that provided data for the control dataset. In some embodiments, the dataset is a dataset of the plurality of datasets. In some embodiments, the dataset is a control dataset. In some embodiments, the machine learning model outputs the amount of blasts in the subject.

In some embodiments, the method is a method of detecting MDS and an amount of blasts above a predetermined threshold indicates the subject suffers from MDS. In some embodiments, the method is a method of detecting leukemia and an amount of blasts above a predetermined threshold indicates the subject suffers from leukemia. In some embodiments, the threshold is 0%. In some embodiments, the threshold is 5%. In some embodiments, the threshold is 9%. In some embodiments, the threshold is 10%. In some embodiments, the threshold is 15%.

In some embodiments, the method further comprises not administering a therapeutic agent to a subject with amounts of blasts below the predetermined threshold. In some embodiments, the method further comprises administering a therapeutic agent to a subject determined to suffer from a pathology of the bone marrow. In some embodiments, the method further comprises administering a therapeutic agent to a subject with amounts of blasts above the predetermined threshold. In some embodiments, the agent is an anticancer agent and the subject suffers from cancer. In some embodiments, the cancer is MDS. In some embodiments, the agent is an anti-MDS agent. In some embodiments, the anti-MDS agent is lenalidomide. In some embodiments, the agent is an anti-leukemia agent. Anticancer agents are well known in the art and any such agent may be used, this includes, but is not limited to, chemotherapy, radiation therapy, immunotherapy, and targeted therapy. In some embodiments, the agent is a chemotherapy. In some embodiments, the agent is radiation therapy. In some embodiments, the agent is an immunotherapy. In some embodiments, the immunotherapy is immune checkpoint inhibition. In some embodiments, the checkpoint is PD-1/PD-L1. In some embodiments, the immunotherapy is CAR-T or CAR-NK therapy. In some embodiments, the anticancer agent is a hypomethylating agent. In some embodiments, the hypomethylating agent is azacytidine. In some embodiments, the hypomethylating agent is decitabine. In some embodiments, the anticancer agent is azacytidine in combination with venetoclax. In some embodiments, the subject suffers from leukemia and the anticancer agent is venetoclax. In some embodiments, the subject suffers from MDS and the agent is azacytidine. In some embodiments, the subject suffers from MDS and the agent is azacytidine in combination with venetoclax. In some embodiments, the leukemia is chronic lymphocytic leukemia, small lymphocytic lymphoma, or acute myeloid leukemia. In some embodiments, the method further comprises performing a bone marrow transplant on a subject determined to suffer from a pathology of the bone marrow. In some embodiments, the method further comprises performing a bone marrow transplant on a subject with an amount of blasts above a predetermined threshold. In some embodiments, the subject suffers from MPN and the agent is an interferon. In some embodiments, the subject suffers from MPN and the method further comprises administering interferon therapy. In some embodiments, the interferon is interferon alpha. In some embodiments, interferon is a type I interferon. In some embodiments, interferon is interferon beta. In some embodiments, interferon beta is interferon beta 1 (IFNB1). In some embodiments, interferon is interferon alpha. In some embodiments, interferon alpha is selected from interferon alpha 1, 2, 4, 5, 6, 7, 8, 10, 13, 14, 16, 17 and 21. In some embodiments, interferon is interferon alpha-2b. In some embodiments, the agent is Ropeginterferon alfa-2b (Besremi).

By another aspect, there is provided a method of calculating a Molecular International Prognostic Scoring System (IPSS-M) risk score for a subject, the method comprising:
a. predicting the percentage of blasts in the bone marrow to the subject by a method of the invention;
b. receiving data as to the presence of bone marrow mutations and/or karyotype abnormalities in the subject;
c. receiving hemoglobin levels and/or platelet counts in peripheral blood from the subject; and
d. calculating the IPSS-M risk score based on the predicted blast percentage, received mutations and/or karyotyping data and received hemoglobin levels and/or platelet counts;
   thereby calculating an IPSS-M risk score.

In some embodiments, the method further comprises detecting the presence of bone marrow mutations. In some embodiments, the method further comprises detecting karyotype abnormalities. In some embodiments, the detecting is in the scRNA data. In some embodiments, the detecting is a non-invasive detecting. In some embodiments, the detecting does not comprise detecting within the bone marrow. It will be understood that all steps of the method can be performed non-invasively and one of the major benefits of the method of the invention is that is does not require a bone marrow sample in order to learn important information (e.g., IPSS-score) about the bone marrow. Methods of karyotyping and performing mutational analysis from scRNA-seq data are described hereinbelow. Further, they have been disclosed in the art, such as in Weissbein et al., “Analysis of chromosomal aberrations and recombination by allelic bias in RNA-Seq”, Nature Communications volume 7, Article number: 12144 (2016), and Petti et al., “A general approach for detecting expressed mutations in AML cells using single cell RNA sequencing”, Nature Communications volume 10, Article number: 3660 (2019), herein incorporated by reference in their entirety.

In some embodiments, the mutation or karyotype abnormality is del(5q). In some embodiments, the mutation or karyotype abnormality is −7/del(7q). In some embodiments, the mutation or karyotype abnormality is −17/del(17p). In some embodiments, the mutation or karyotype abnormality is a complex karyotype. In some embodiments, the mutation or karyotype abnormality is del(11q). In some embodiments, the mutation or karyotype abnormality is del(5q). In some embodiments, the mutation or karyotype abnormality is del(12p). In some embodiments, the mutation or karyotype abnormality is del (20q). In some embodiments, the mutation or karyotype abnormality is del (7q). In some embodiments, the mutation or karyotype abnormality is +8. In some embodiments, the mutation or karyotype abnormality is +19. In some embodiments, the mutation or karyotype abnormality is i(17q). In some embodiments, the mutation or karyotype abnormality is −Y. In some embodiments, the mutation or karyotype abnormality is −7. In some embodiments, the mutation or karyotype abnormality is (inv)3/t(3q)/del(3q).

In some embodiments, the mutation is a variant allele. In some embodiments, the mutation is mutation within tumor protein p53 (TP53). In some embodiments, mutation is the number of mutations. In some embodiments, the mutation or karyotype abnormality is loss of heterozygosity of the TP53 locus. In some embodiments, the mutation is MLL (lysine methyltransferase 2A (KMT2A)) mutation. In some embodiments, the mutation is fms related receptor tyrosine kinase 3 (FLT3) mutation. In some embodiments, the mutation is ASXL transcriptional regulator 1 (ASXL1) mutation. In some embodiments, the mutation or karyotype abnormality is Cbl proto-oncogene (CBL) mutation. In some embodiments, the mutation is DNA methyltransferase 3 alpha (DNMT3A) mutation. In some embodiments, the mutation is ETS variant transcription factor 6 (ETV6) mutation. In some embodiments, the mutation is Enhancer Of Zeste 2 Polycomb Repressive Complex 2 Subunit (EZH2) mutation. In some embodiments, the mutation is isocitrate dehydrogenase (NADP(+)) 2 (IDH2) mutation. In some embodiments, the mutation is KRAS proto-oncogene, GTPase (KRAS) mutation. In some embodiments, the mutation is nucleophosmin 1 (NPM1) mutation. In some embodiments, the mutation is NRAS proto-oncogene, GTPase (NRAS) mutation. In some embodiments, the mutation is RUNX family transcription factor 1 (RUNX1) mutation. In some embodiments, the mutation is splicing factor 3b subunit 1 (SF3B1) mutation. In some embodiments, the mutation is serine and arginine rich splicing factor 2 (SRSF2) mutation. In some embodiments, the mutation is U2 small nuclear RNA auxiliary factor 1 (U2AF1) mutation. In some embodiments, the mutation is BCL6 corepressor (BCOR) mutation. In some embodiments, the mutation is BCL6 corepressor like 1 (BCORL1) mutation. In some embodiments, the mutation is CCAAT enhancer binding protein alpha (CEBPA) mutation. In some embodiments, the mutation is ethanolamine kinase 1 (ETNK1) mutation. In some embodiments, the mutation is GATA binding protein 2 (GATA2) mutation. In some embodiments, the mutation is G protein subunit beta 1 (GNB1) mutation. In some embodiments, the mutation is isocitrate dehydrogenase (NADP(+)) 1 (IDH1) mutation. In some embodiments, the mutation is neurofibromin 1 (NF1) mutation. In some embodiments, the mutation is PHD finger protein 6 (PHF6) mutation. In some embodiments, the mutation is protein phosphatase, Mg2+/Mn2+ dependent 1D (PPM1D) mutation. In some embodiments, the mutation is pre-mRNA processing factor 8 (PRPF8) mutation. In some embodiments, the mutation is protein tyrosine phosphatase non-receptor type 11 (PTPN11) mutation. In some embodiments, the mutation is SET binding protein 1 (SETBP1) mutation. In some embodiments, the mutation is STAG2 cohesin complex component (STAG2) mutation. In some embodiments, the mutation is WT1 transcription factor (WT1) mutation.

In some embodiments, hemoglobin levels are received. In some embodiments, the method further comprises measuring hemoglobin levels. In some embodiments, the method comprises receiving a blood sample from the subject. In some embodiments, the hemoglobin levels are calculated in the blood sample. In some embodiments, platelet counts are received. In some embodiments, the method further comprises counting platelets. In some embodiments, the platelets are in the blood sample. In some embodiments, the method further comprises receiving neutrophil counts. In some embodiments, the method further comprises counting neutrophils. In some embodiments, neutrophils in the sample are counted. In some embodiments, the subject's age is also received. In some embodiments, the subject's sex/gender is also received.

In some embodiments, the IPSS-M risk score is calculated based on any combination of received data. In some embodiments, the IPSS-M risk score is calculated based on the predicted blast percentage. In some embodiments, the IPSS-M risk score is calculated based on the predicted blast percentage and received mutations and karyotyping. In some embodiments, the IPSS-M risk score is calculated based on the predicted blast percentage and the received hemoglobin levels and platelet counts. In some embodiments, the IPSS-M risk score is calculated based on the predicted blast percentage, received mutations and karyotyping and received hemoglobin levels and platelet counts. In some embodiments, the IPSS-M risk score is calculated further based on the neutrophil counts and/or the patient's age.

The IPSS-M score is well known in the art. It ranges from 0 to 16. The scores are divided into six risk possibilities: Very Low (VL) risk, Low (L) risk, Medium Low (ML) risk, Medium High (MH) risk, High risk (H) and Very High (VH) risk. Subjects with low risk may receive no treatment or treatment to manage symptoms such as Erythropoiesis-stimulating agents (ESA) to treat anemia. Patients with thrombocytopenia may receive romiplostim or eltrombopag. Similarly, Luspatercept can be administered if ESA is ineffective (and/or there is a mutation in SF3B1 or ring sideroblasts are present). Subjects with high risk may receive hypomethylating agents, or other anticancer treatments. High risk subjects may have a bone marrow transplant.

In some embodiments, the method further comprises administering to a subject a treatment regimen based on the calculated IPSS-M score. In some embodiments, a subject with a higher score is administered a more intense treatment regimen. In some embodiments, a subject with a lower score is administered a reduced treatment regimen. In some embodiments, more intense is increased. In some embodiments, reduced is less intense.

By another aspect, there is provided a method of detecting AML in a subject, the method comprising detecting the presence of an R353K mutation within GATA3 in a sample from the subject, thereby detecting AML in a subject.

In some embodiments, the sample comprises cells. In some embodiments, the cells are hematopoietic cells. In some embodiments, the cells are blasts. In some embodiments, the cells are CD34 positive cells. In some embodiments, mutation is a mutation of arginine 353 in GATA3. In some embodiments, the arginine is mutated to lysine. In some embodiments, the mutation is indicative of AML.

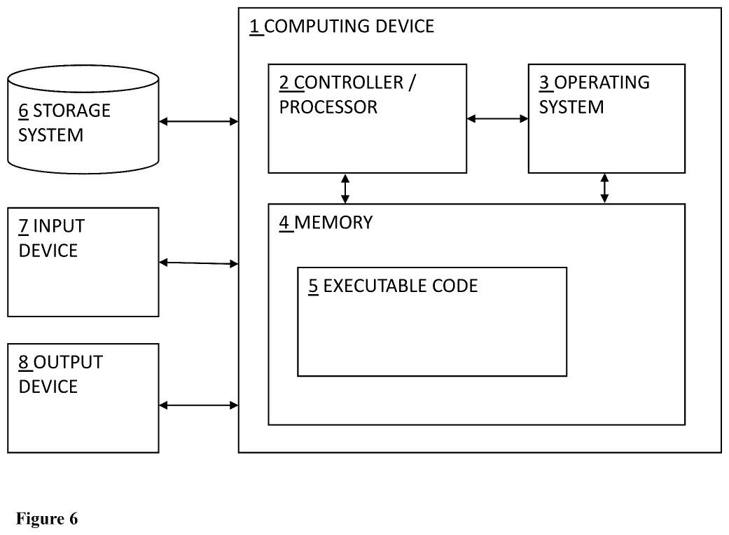

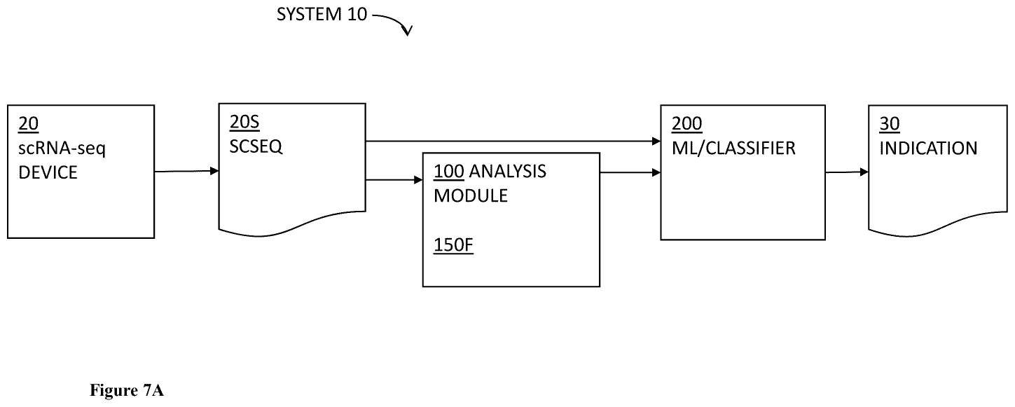

Reference is now made to FIG. 6, which is a block diagram depicting a computing device, which may be included within an embodiment of a system for analyzing bone marrow or calculating an IPSS-M risk score, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may calculate an IPSS-M score for a subject as further described herein. Although, for the sake of clarity, a single item of executable code 5 is shown in FIG. 6, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to single cell RNA sequencing (scRNA-seq) reads may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

The term neural network (NN) or artificial neural network (ANN), e.g., a neural network implementing a machine learning (ML) or artificial intelligence (AI) function, may be used herein to refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. At least one processor (e.g., processor 2 of FIG. 6) such as one or more CPUs or graphics processing units (GPUs), or a dedicated hardware device may perform the relevant calculations.

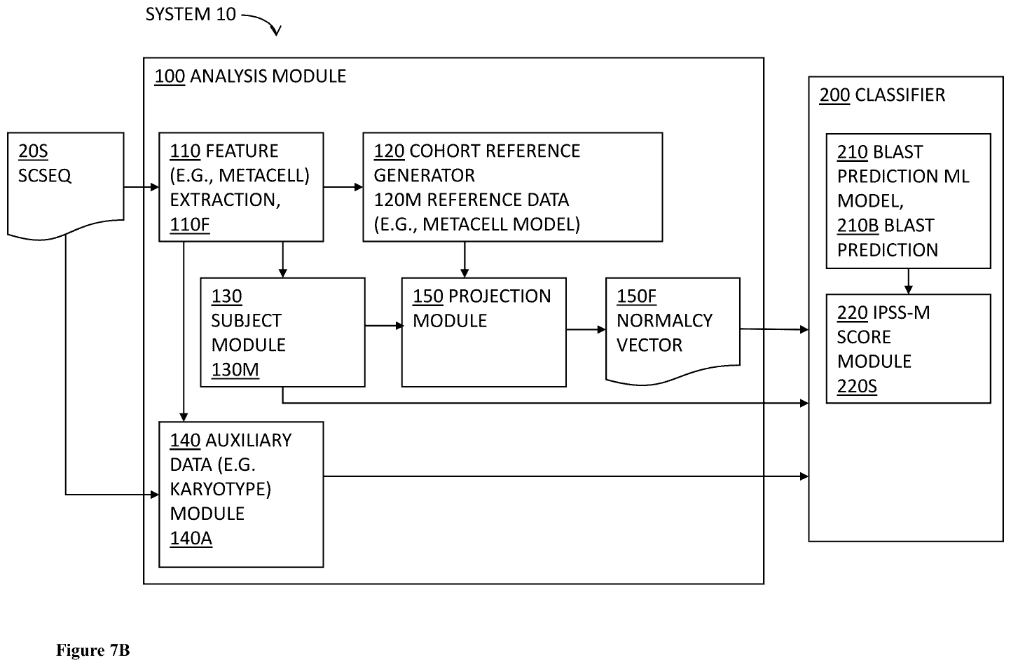

Reference is now made to FIG. 7, which depicts a system 10 for analyzing bone marrow in a subject, according to some embodiments.

According to some embodiments of the invention, system 10 may be implemented as a software module, a hardware module, or any combination thereof. For example, system may be or may include a computing device such as element 1 of FIG. 6 and may be adapted to execute one or more modules of executable code (e.g., element 5 of FIG. 6) to analyze bone marrow in a subject, as further described herein.

As shown in FIG. 7, arrows may represent flow of one or more data elements to and from system 10 and/or among modules or elements of system 10. Some arrows have been omitted in FIG. 7 for the purpose of clarity.

In some embodiments, analyzing comprises producing a feature vector representing deviation of the subject's cellular data from the control cellular data.

As shown in FIG. 7A, system 10 may include, or may be communicatively connected to a single cell RNA sequencing (scRNA-seq) module or device 20, which may be configured to produce scRNA-seq data 20S (or “data 20S”, for short) as elaborated herein.

An analysis module 100 of system 10 may be configured to analyze data 20S, to extract a feature vector 150F. As elaborated herein, feature vector 150F may include one or more values indicative of a CD34 positive population in a peripheral blood sample of a subject (e.g., patient) of interest.

For example, feature vector 150F may include a plurality of entries, each corresponding to a specific cell type. The value of each entry of feature vector 150F may represent a relation to, or deviation from a reference value, or a range of cell populations.

Referring to the example of FIG. 2D (top panel), the reference values for a range, and mean of a frequency of each type of stem cell population may be indicated by the gray, and dashed lines. In such embodiments, entries of a feature vector 150F pertaining to a specific subject (e.g., #115, green line) may include values of stem cell population of that subject. Additionally, or alternatively, entries of a feature vector 150F may include statistical numerical values representing deviation from a reference. Such a reference may include a mean (black, dashed line) and/or normal range (grey lines) of stem cell population in a cohort of subjects.

In some embodiments, analyzing comprises applying a trained Machine Learning (ML) based module 200, also referred to herein as a classifier 200, to the received dataset 20S. Additionally, or alternatively, analyzing may include applying ML 200 on feature vector 150F. In some embodiments, the ML module is trained on a training set. In some embodiments, the training set comprises the control dataset. In some embodiments, the training set comprises the plurality of cellular datasets.

In some embodiments, the ML 200 may output (e.g., via output device 8 of FIG. 6) an indication 30. Indication 30 may be, for example, a classification of the subject's bone marrow. In some embodiments, indication 30 may include a classification of the subject, an analysis of the subject's bone marrow. Additionally, or alternatively, indication 30 may include a notification regarding a health condition of the subject (e.g., healthy, or not), a diagnosis of the subject (e.g., a suspected pathology of the bone marrow), a prognosis of a subject's condition, and the like.

In some embodiments, analyzing may include applying ML 200 to a parameter extracted from the dataset. In some embodiments, analyzing comprises applying a trained machine learning model to feature vector 150F.

Reference is now made to FIG. 7B, which is a block diagram depicting a non-limiting example for implementation of system 10, according to some embodiments of the invention. System 10 of FIG. 7B may be the same as system 10 of FIG. 7A.

As shown in FIG. 7B, analysis module 100 may include a feature extraction module 110. As elaborated herein, feature extraction module 110 may be configured to extract, from data 20, a plurality of features 110F, or parameters pertaining to, or characterizing of a plurality of specific cells in peripheral blood samples. These features may be expression profiles of informative genes or other transcriptional data extracted from the scRNA-seq data. The features may be the whole transcriptome or informative parts of the transcriptome of the cells.

Analysis module 100 may use features 110F to bin, or cluster features 110F to form high-level representations of cell population in the peripheral blood samples.

For example, a subject module 130 of analysis module 100 may be configured to produce at least one subject-specific model 130M. Subject-specific model 130M may pertain to a specific peripheral blood test, taken from a specific subject. In some embodiments, subject-specific model 130M may include a plurality of metacell entities, each representing an abstraction of cell population data pertaining to that subject, as elaborated herein.

Additionally, or alternatively, a cohort reference generator module 120 of analysis module 100 may be configured to produce a reference data 120M, or cohort data model 120M, also referred to herein as an HSPC atlas 120M. In some embodiments, reference data 120M may include a plurality of metacell entities, each representing an abstraction of cell population data pertaining to a cohort of subjects, as elaborated herein.

As shown in FIG. 7B, analysis module 100 may include a projection module 150, configured to project, or compare features 110F of a specific subject of interest, as manifested by subject-specific model 130M, onto features of the cohort of subjects, as manifested by reference data (e.g., HSPC atlas) 120M.

According to some embodiments, based on this comparison or projection, projection module 150 may produce a feature vector 150F, also denoted herein as a “normalcy vector” 150F. Normalcy vector 150F may be indicative of the specific subject's condition.

According to some embodiments, system 10 may infer classifier 200 on feature vector (e.g., normalcy vector) 150F, to produce indication 30 of FIG. 7A. In such embodiments, classifier 200 may be, or may include an ML-based classification model, that may be trained on a training dataset, that includes a plurality of labeled, or annotated normalcy vectors 150F. Annotation of normalcy vectors 150F may include, for example, an expert indication 30 (e.g., diagnosis) of corresponding peripheral blood samples. ML-based classification model 200 may be trained to produce indication 30 of incident normalcy vectors 150F by a supervised training scheme, using the annotations as supervisory data.

Additionally, or alternatively, system 10 may infer classifier 200 on subject-specific model 130M data, to produce indication 30. In such embodiments, classifier 200 may be, or may include an ML-based classification model, that may be trained on a training dataset, that includes a plurality of labeled, or annotated subject-specific model 130M data entities. Annotations of subject-specific models 130M of the dataset may include, for example, expert indications 30 (e.g., diagnosis) of corresponding peripheral blood samples. ML-based classification model 200 may thus be trained to produce indication 30 by a supervised training scheme, using the annotations as supervisory data.

Additionally, or alternatively, system 10 may infer classifier 200 on feature vector (e.g., normalcy vector) 150F, to produce a prediction of blast level 210B in bone marrow. In such embodiments, classifier 200 may be, or may include an ML-based classification model 210, that may be trained on a training dataset, that includes a plurality of labeled, or annotated normalcy vectors 150F. Annotation of normalcy vectors 150F may include levels of blasts 210B in bone marrows, corresponding to respective patient peripheral blood samples. ML-based classification model 210 may be trained to predict bone marrow blast levels 210B by a supervised training scheme, using the annotations as supervisory data.

Additionally, or alternatively, system 10 may include an auxiliary data extraction module 140 (or “auxiliary module 140” for short). For example, auxiliary module 140 may be configured to produce, from data 20, auxiliary information 140A such as karyotype data 140A or mutational data, as known in the art. In such embodiments, classifier module 200 may include an IPSS-M risk score calculation module 220, configured to calculate an IPSS-M risk score 220S based on the predicted bone-marrow blast level 210B, the calculated karyotype data 140A, mutational data and other clinical blood measurements, as known in the art.

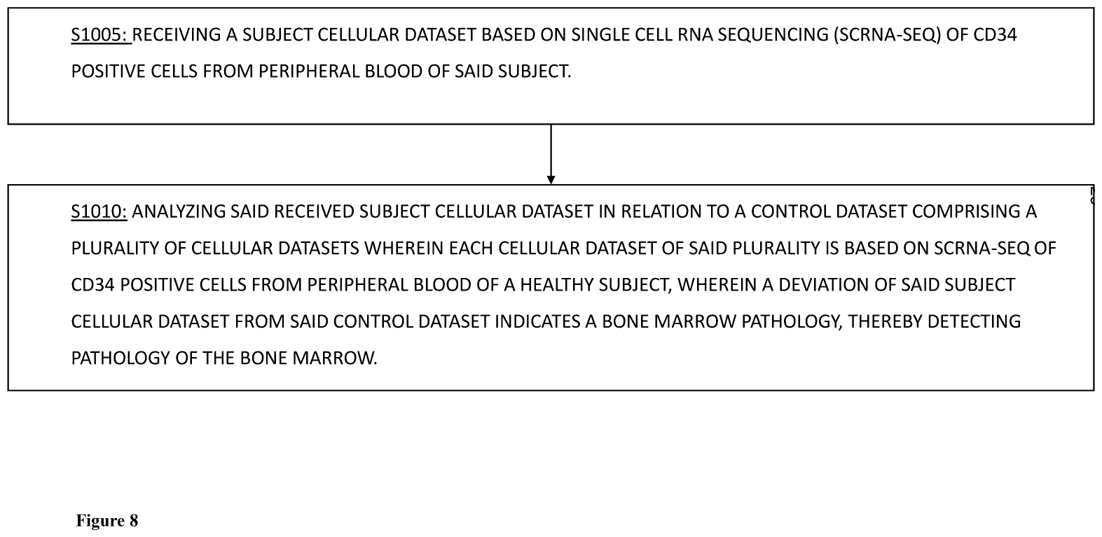

Reference is now made to FIG. 8, which is a flow diagram depicting a method of analyzing bone marrow in a subject, by at least one processor, according to some embodiments.

As shown in step S1005, the at least one processor (e.g., processor 2 of FIG. 6) may receive a subject cellular dataset 20S based on single cell RNA sequencing (scRNA-seq) 20 of CD34 positive cells from peripheral blood of the subject.

As shown in step S1010, the at least one processor may employ an analysis module 100 (e.g., as elaborated herein in relation to FIGS. 7A, 7B), to analyze said received subject cellular dataset (e.g., 130M) in relation to a control dataset (e.g., 120M) comprising a plurality of cellular datasets. Each cellular dataset of said plurality may be based on scRNA-seq 20S of CD34 positive cells from peripheral blood of a healthy subject, wherein a deviation (e.g., feature vector, or normalcy vector 150F) of said subject cellular dataset from said control dataset may indicate a bone marrow pathology. Embodiments of the invention may thereby produce an indication 30, representing, or notifying detection of pathology of the bone marrow in the subject.

By another aspect, there is provided a system for performing a method of the invention.

In some embodiments, the system is for evaluating bone marrow healthy. In some embodiments, the system is for measuring blast number in the bone marrow. In some embodiments, the system is a non-invasive system.

In some embodiments, the system comprises a scRNA sequencing device. In some embodiments, sequencing device is a scRNA sequencer. In some embodiments, the system comprises a non-transitory memory device, wherein modules of instruction code are stored. In some embodiments, the system comprises at least one processor. In some embodiments, the processor is associated with the memory device. In some embodiments, the processor is configured to perform a method of the invention. In some embodiments, the processor is configured to execute the modules of instruction code, whereupon execution of said modules of instruction code, the at least one processor is configured to perform a method of the invention.

In some embodiments, the method comprises obtaining from the scRNA sequencing device single cell transcriptomes from CD34 positive cells from peripheral blood. In some embodiments, the peripheral blood is from the subject. In some embodiments, the method comprises producing a cellular dataset from the obtained single cell transcriptomes. In some embodiments, the method comprises producing a cellular dataset based on the obtained single cell transcriptomes. In some embodiments, the method comprises producing a cellular dataset derived from the obtained single cell transcriptomes. In some embodiments, the method comprises analyzing the produced dataset. In some embodiments, the analyzing is in relation to a control dataset. In some embodiments, the method comprises accessing a control dataset. In some embodiments, the control dataset is a control database. In some embodiments, the control dataset is a plurality of datasets. In some embodiments, the method comprises outputting a finding. In some embodiments, the finding is the health of the subject. In some embodiments, the finding is the health of the bone marrow. In some embodiments, the finding is healthy. In some embodiments, the finding is the presence of bone marrow pathology. In some embodiments, the finding is what the bone marrow pathology is. In some embodiments, the finding is based on deviation or lack thereof of the subject dataset from the control dataset.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising,” otherwise analogous embodiments described in terms of “consisting of” and/or “consisting essentially of” are included.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J.E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization-A Laboratory Course Manual” CSHL Press (1996); all of which are incorporated by reference. Other general references are provided throughout this document.

### Materials and Methods

Sample procurement and handling: Fresh peripheral blood samples were collected from 148 healthy individuals (79 males, 69 females) aged 23-91. All sample donors were considered healthy, their CBCs were within normal range, and they were not known to have any CH defining mutations prior to sequencing. Written informed consent allowing access to longitudinal CBCs and sequencing data (CH and genotyping panels) was obtained from all participants in accordance with the Declaration of Helsinki. All relevant ethical regulations were followed, and all protocols were approved by the Weizmann Institute of Science ethics committee (under IRB protocol 283-1).

Recruitment was intended to allow characterization of the normal variation in cHSPC states. As no such profiling had been previously performed, not much could be assumed regarding the variance in the population a-priori. The aim was therefore to profile responsive volunteers with normal blood counts, balancing sex and seeking a dispersed age distribution biased toward older individuals. This strategy was reassessed following initial sampling, and observed remarkable homogeneity in transcriptional states across individuals sampled from the immediate community as well as from HMO out-patient clinics, emergency medical centers, hospital wards etc. This was critically important, as it confirmed the universality of the model across individuals.

50 ml of peripheral blood (PB) were drawn from each individual into lithium-heparin tubes. 1 ml of blood was used for DNA production, and the remaining volume was used for PBMC isolation via Ficoll, using Lymphoprep filled Sepmate tubes (StemCell technologies), followed by CD34 magnetic bead-based enrichment using the EasySep human CD34 positive selection kit II (StemCell technologies). This enrichment strategy was found to be simple and reproducible and it was chosen for several reasons: 1) RNA-seq data was most reproducible when cells were not sorted, but rather enriched-for using beads (lower mitochondrial gene fraction). 2) CD34 purity could be highly regulated by this method, to achieve anywhere between 50-95% enrichment of CD34-positive cells, which could later be easily distinguished based on their single cell expression data. In terms of cell numbers—50 ml of blood would yield anywhere between 50 to 100 million PBMCs following Ficoll, 1/1000 of which are expected to be CD34+, such that this population's representation was increased from 0.1% in the periphery to at least 50% of cells loaded for analysis.

scRNA-seq of CD34+ PBMCs: Single cell RNA libraries were generated using the 10× genomics scRNA-seq platform (Chromium Next Gem single cell 3′ reagent kit V3.1). Chip loading was preceded by flow-cytometry to verify that enrichment was successful, and that enough CD34+CD45^{int} live cells were gathered. All blood samples were freshly drawn at the Weizmann Institute of Science on the morning of each experiment day, and time from blood draw to 10× loading was restricted to 5 hours. The motivation for working with fresh samples was based on previous experience with PB CD34+ cells being vulnerable to freezing/thawing rounds and long manipulation times.

All 10× libraries were sequenced on two alternative platforms (Illumina/Ultima Genomics). 12 libraries were simultaneously sequenced on both platforms for comparison purposes and in order to demonstrate the scalability of the approach. It was observed that the Ultima-sequenced data was highly similar to the Illumina-sequenced data.

Genotype-based demultiplexing: All cells were traced back to their sample of origin using genotype-based de-multiplexing. This method allowed pooling of blood samples immediately following extraction of the DNA aliquot, such that CD34-enrichment was performed on the entire pool of PBMCs produced. The use of SNP-based multiplexing has several advantages to alternative antibody-based cell hashing methods: 1) it is extremely cost effective, such that the cost of sequencing a single individual on a 2000 SNP Molecular Inversion Probe (MIP) panel at a depth of 1000× per SNP (adequate for de-multiplexing purposes) is several folds cheaper than antibody staining, 2) genotyping eliminates the need to keep samples separated prior to loading, it entails shorter handling times and less cell manipulation, as it does not require antibody incubation periods and multiple wash centrifuges. This was very evident in cell viability prior to chip loading. As with other methods of sample multiplexing, genotype-based multiplexing allows for robust doublet detection during data analysis, which enabled loading of 30-40K cells from between 4-6 individuals on each Chromium Chip lane, yielding 15-25k cells per library.

Molecular inversion probe (MIP) panels: Both the CH and genotyping panels are Molecular inversion probes (MIP)-based panels described in detail previously in Biezuner, T. et al., “An improved molecular inversion probe based targeted sequencing approach for low variant allele frequency.” NAR Genom Bioinform 4, (2022) herein incorporated by reference in its entirety. The CH panel contains 705 probes, covering pre-leukemic SNVs and Indels in 47 genes, and is complemented by 2 amplicon sequencing reactions to cover GC rich regions in SRSF2 and ASXL1. As MIP sequencing is cost-effective yet noisy, an in-house variant-calling method was designed to identify low VAF CH events. It is described in Biezuner, et al. “An improved molecular inversion probe based targeted sequencing approach for low variant allele frequency”, NAR Genom Bioinform 4, (2022), the contents of which are hereby incorporated by reference in their entirety. The genotyping panel allows for the simultaneous detection of >2000 common genetic variants, all of which are extensively covered in all cell types in the data. It includes heterozygous sites with at least 5% minor allele frequency from the 1K genomes project, which were highly covered by RNA molecules in the data (at least 80 UMIs across all cells in a test 10× library), excluding sites in repetitive elements and in sex chromosomes. Both panels were designed using MIPgen to ensure capture uniformity and specificity.

CH sequencing of high RDW samples and controls: In order to compare propensity for CH and high risk CH mutations in high RDW cases and normal RDW controls, deep targeted sequencing was performed on DNA samples from 602 high RDW (>15%) individuals, who did not show signs of anemia and whose blood count did not meet MDS criteria (11.5 g/dL≤Hg≤15.5 g/dL [F], 13 g/dL≤Hg≤17 g/dL [M], 80 fL≤MCV≤96 fL, PLT≥100×109/L, Abs Neut≥1.8×109/L), and 602 normal RDW (11.5 g/dL≤Hg≤15.5 g/dL [F], 13 g/dL≤Hg≤17 g/dL[M], 80 fL≤MCV≤96 fL, PLT≥100×109/L, Abs Neut≥1.8×109/L), age and gender-matched controls. Case-Control matching was performed using the R MatchIt package, balanced on age and sex, method=“nearest”, ratio=1, from a total of 18,147 individuals with longitudinal blood counts and available DNA. All DNA samples were collected after obtaining written informed consent and in accordance with the Declaration of Helsinki and were received de-identified from the Tel Aviv Sourasky Medical Center (TASMC) Integrative Cancer Prevention Clinic. All relevant ethical regulations were followed, and all protocols were approved by the TASMC ethics committee (under IRB protocol 02-130).

scRNA-seq processing: fastq files were processed by executing cellranger-with an hg-38 reference genome. Cells were filtered with at least 20% mitochondrial expression and ≤500 UMIs from unfiltered genes.

Doublet calling: Several steps were performed to assign cells to their individuals and to detect doublets. The pipeline is made of several steps:
1. Demultiplexing cells and calling doublets based on SNPs found in the scRNAseq data;
2. Building a metacell model using cells from all libraries, including cells previously marked as doublets, identifying and removing metacells made of doublets;
3. Identifying doublet metacells based on expression of marker genes;
4. Building the final metacell model and marking metacells as doublets based on expression markers.

In the first step, doublets were identified and cells assigned to individuals using Vireo and Souporcell, which cluster cells based on SNPs found in sequenced RNA molecules. Vireo (preceded by running cellsnp) and Souporcell were executed on each library separately. Both methods used SNPs from the genotyping panel which were covered by at least 20 UMIs in the library (in Souporcell—at least 10 from the major and minor allele each). High agreement was observed in doublet calling between the two methods.

In the next step, a metacell model was built with cells from all libraries. This model included cells that were already identified as doublets. The model was built with metacell (see Lee-Six, H. et al., “Population dynamics of normal human blood inferred from somatic mutations.” Nature 561, 473-478 (2018), herein incorporated by reference in its entirety), with a target metacell size of 200 cells. All metacells where at least 35% of the cells were already marked as doublets were then marked, and all metacells that expressed key markers of distinct cell types, as doublet metacells. All cells that belonged to a doublet metacell were then marked as doublets. An additional metacell model (see below) was then built, without cells that were marked as doublets.

Correcting for sequencing platform bias: Few of the 10× libraries were sequenced on an Ultima Genomics sequencer, and as most libraries were processed through a standard Illumina pipeline, it was wished to minimize batch effects related to these sequencing platform variations. To this end, libraries that were sequenced on both platforms were used to calculate an Illumina-Ultima correction factor per gene as the mean log2-fold change in expression of the gene across re-sequenced libraries. Each Ultima-sequenced library was then normalized by downsampling genes with at least 0.28 log2-fold Ultima overexpression, and resampling genes with at least 0.2 Illumina overexpression. The downsampling and resampling were performed for each gene independently, across all cells in each Ultima library. The thresholds for downsampling and resampling were chosen such that the overall number of UMIs per cells remained similar. 87 genes with at least 4-fold change between Ultima and Illumina were excluded from further processing.

Computing the reference metacell model: The metacell model was built with metacell 2, with a target metacell size of 200 cells. Histone, cell cycle, ribosomal, sex-linked, and stress response genes (including FOS, JUN) were marked as forbidden genes, as were genes with high technical variation, such as those with high or inconsistent differences between Illumina-and Ultima-sequenced technical replicates. These genes were not used for calculating gene-gene similarities but were included in downstream analyses. Metacells were annotated using known markers. Metacells with low CD34 expression, such as mature monocytes, B cells, T cells, NK cells, DCs, and endothelial cells were excluded from most downstream analyses. UMAP projections of the metacell expression vector over genes with specific enrichment over cell types were used for visualization of the metacell manifold.

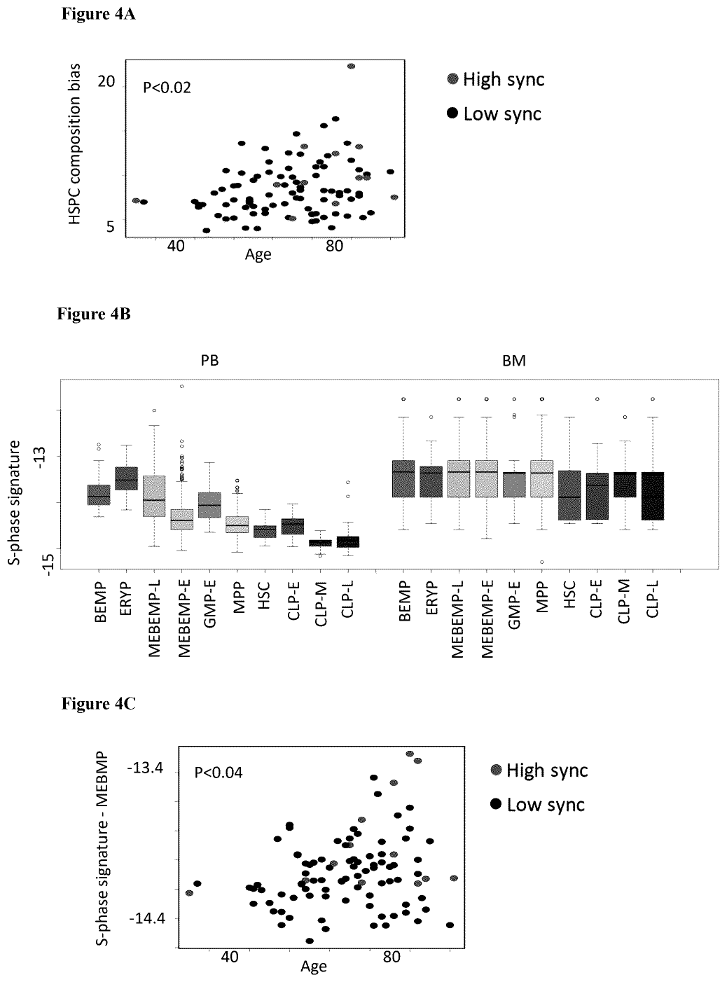

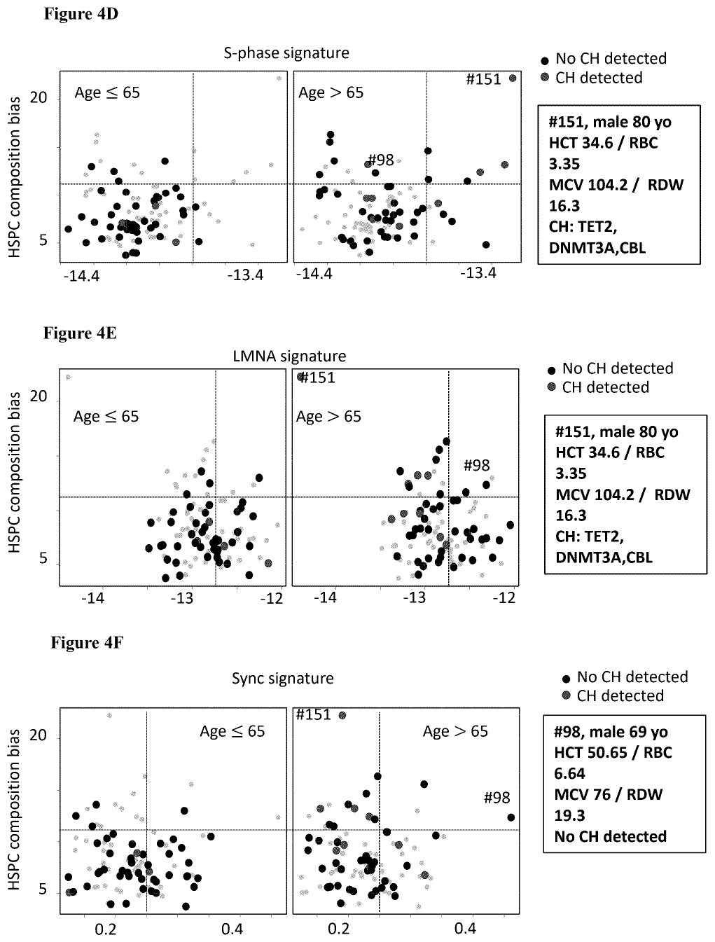

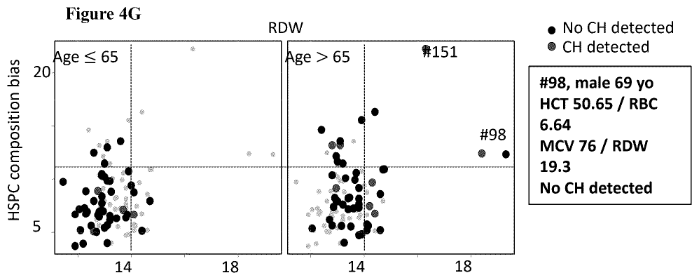

BM comparisons and projections: Three BM datasets were used for comparison purposes: a dataset including CD34-enriched cells from 2 individual BMs collected by us and processed similarly to PB (FIG. 1A); the Human Cell Atlas (HCA) dataset and a CD34+ bead-enriched BM datasets from Setty, et al. “Characterization of cell fate probabilities in single-cell data with Palantir”, Nat Biotechnol 37, 451-460 (2019), the contents of which are hereby incorporated by reference in their entirety. The HCA datasets was previously processed and annotated in a metacell model. A metacell model for the 2 CD34-enriched BM samples collected was constructed using metacell, in a similar fashion to that described previously, and the Setty et al. sequencing data was downloaded and processed by running cellranger and a third BM metacell model was created from that data. To project both the PB data and the Setty dataset on the HCA dataset, the HCA metacells and the Setty and PB metacells were correlated over genes showing high variance over the HCA metacell model. Each Setty metacell was annotated using the mode of the 5 most correlated HCA metacells and expression of gene markers. Metacells from each of these models was projected on the HCA UMAP using the mean x and y values of its 5 most correlated HCA metacells. To compare S-phase genes between the PB and BM (FIG. 4B), there was calculated the S-phase signature (mean expression of six cell cycle genes: Thymidylate synthase (TYMS), H2A.Z Variant Histone 1 (H2AZ1/H2AFZ), Proliferating Cell Nuclear Antigen (PCNA), Minichromosome Maintenance Complex Component 4 (MCM4), helicase, lymphoid specific (HELLS), and marker of proliferation Ki-67 (MKI67)) for each PB and HCA metacell and plotted the distribution of these scores across metacells for each cell type.

HSC differentiation gene programs: To visualize transcriptional dynamics in HSC cells, MEBEMP and CLP metacells were sorted based on their AVP expression. To calculate differential expression (DE) between HSC and neighboring cell types, the geometric mean of each gene was calculated across HSCs, CLP-E and MPP metacells, and the difference between HSC and MPP, and between HSC and CLP-E was selected.

Differential expression between individuals unexplained by the metacell model: Each individual's pooled expression profile was compared to a matched expression profile based on the individual's distribution across metacells. The analysis was performed separately for MPP/MEBEMPs (BEMP, ERYP, MEBEMP-E/L, GMP-E and MPP) and CLPs (CLP-E/M/L, NKTDP). In each group of cell types, each cell was downsampled to have 500 UMIs and the UMIs across all cells of each individual were summed, the sum was normalized to 1 and log2 was calculated, to obtain the observed expression. To compute matched expression, each metacell was downsampled to have 90K UMIs and all UMIs of the metacell each cell belongs to were summed for each individual. This matched expression was normalized to sum to 1 and log2 was calculated. All genes that were expressed in either the observed or matched expression in any individual (log2 expression>2\hat{ }−14.5), with at least a 2-fold change between observed and matched in at least one individual were plotted. Genes exhibiting strong batch effects were excluded.

HSPC compositional analysis: To explore variance in cell type composition between individuals, first the distribution of each individual's cells across the CD34+ cell states were calculated. Further, cells from CD34+ states were partitioned into finer grained bins using one HSC bin, four CLP bins, and ten MEBEMP/MPP bins, for a total of 15 bins. HSC cells were assigned to bin 0, CLP-E cells to CLP bin 1, and CLP-M/L cells to CLP bins 2-4 based on an AVP expression gradient, such that each of these bins consisted of an equal number of cells. Similarly, MPP and MEBEMP-E/L cells were assigned into equal size MPP/MEBEMP bins 1-10 based on decreasing AVP expression.

The bottom panel of FIG. 2D, shows individual enrichment across bins (log2 of the ratio each individual's cell frequency in each bind to the median cell frequency in that bin across individuals). Individuals were partitioned into three group based on their mean enrichment across CLP bins 2-4—those with mean enrichment >0.5 are high CLP, those with <−0.5 are low, and the rest are intermediate. Next, the stemness score was defined as the ratio between the number of cells in MPP/MEBEMP bins 1-5 and the total MPP/MEBEMP number (cells in bins 1-10). Individuals with stemness score >0.5 had enriched stemness. Individuals within each cluster were further sorted based on their stemness score. The combination of CLP enrichment and stemness defines the six classes shown in the figure.

Test for association between cell state compositions and a numerical label: Permutation tests were used to test the association between cell state distribution and a label, such as CBC indices or sync-scores. We sorted CD34+ cell states into 11 bins from late MEBEMP differentiation through HSCs to late CLP differentiation (as ordered in FIG. 2B). Triplets of adjacent cell types in this order were examined, and total individual cell state frequency for each triplet was calculated, obtaining 9 vectors of length 150. Then, each of these 9 vectors was correlated to the label vector and the maximal absolute correlation value was taken as a test statistic. This process was repeated after permuting the label 10000 times and the test statistics from the permutations was used to derive a p-value.

Variably expressed gene modules: Genes modules with high variance were detected across individuals while controlling or compositional variant. This was performed, separately for myeloid and lymphoid states, in the following manner:

A) For each individual—the 5^{th} percentile of his/her number of UMIs were calculated across all MPP metacell cells, and all cells were downsampled to this number. Then, all downsampled cells were pooled, normalized to sum to 1 and log2 was calculated. This gave the observed expression profile of each individual.

B) The expected expression profile for each individual was then created as follows: all MPP metacells were partitioned into 30 equal size bins based on their AVP expression, and metacells were downsampled to 90K UMIs. The average expression of each gene across downsampled metacells in each bin was calculated. This defined an expression profile for each of the 30 bins. To obtain an individual's expected expression, the weighted average expression profl of the bins was calculated, where the weight of each bin is proportional to the fraction of the individual's cells from that bin, normalized to sum to 1 and the log2 was calculated. The difference between the observed and expected expression profiles was then calculated.

C) The data showed some batch effect distinguishing samples collected in two calendaric periods. As this effect could introduce co-variation between genes across individuals, a correction controlling for it was applied. This was performed using a linear model fitting each gene to the sample collection period. The inferred period factor was then subtracted from the samples that were collected in the second period. This approach was found to significantly reduce emergence of gene clusters linked with sample collection date bias.

D) Genes with high variance that were unlikely to be affected residually by the main manifold differentiation process were screened for. Genes with high batch effects (Kruskal-wallis p-value <1e−3 when using an individual's 10× batch as a covariate), genes with high AVP correlation (absolute value Pearson correlation >0.65) and genes highly correlated (absolute value Pearson correlation >0.5) were removed with a module of differentially expressed genes between the first and second collection periods. Each gene's variance was then calculated in the difference between the observed and expected expression across individuals. As some of this variance can be explained due to sampling noise, each gene's variance was plotted across individuals against its mean expression across individuals. Genes were sorted by this expression value and from the variance of each gene a rolling mean of the variances of 100 neighboring genes in that ordering was subtracted. Genes with variance at least 0.08 higher than the rolling mean variance were chosen.

E) A gene-gene Spearman correlation matrix was calculated for high variance genes and the correlation profiles were clustered using hierarchical clustering. Genes with low mean correlation (<0.2) to their cluster were removed, and then removed gene clusters with low mean correlation between their genes (<0.25 mean correlation for all gene pairs). Gene-gene correlations were further computed using only samples from the first library collection period and gene clusters were required to have a high mean correlation (>0.25) between their genes when using only these samples. Additional gene modules arising from this analysis were removed due to batch effects or traces of MEBEMP differentiation not normalized by this approach. This resulted in FIG. 3F.

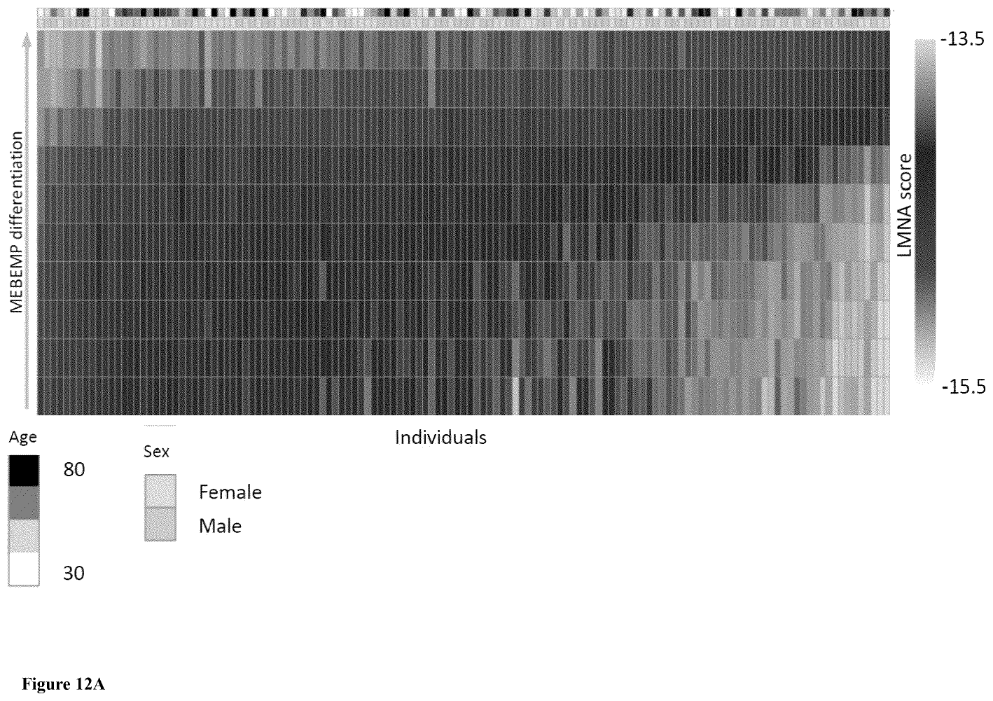

A similar analysis was performed for CLPs (FIG. 11), with few differences. The analysis included cells from CLP-M metacells. The cells were partitioned into 6 equal size bins, and partitioning was based on the average of their DNTT and VPREB1 expression. Genes with high absolute correlation to the average DNTT and VPREB1 expression were excluded. This was followed by hierarchical clustering of the gene-gene correlation profiles, and removal of genes as described for MEBEMPs.

Age regression: Age regression models were developed for MEBEMP and CLP expression separately. To predict age, the difference between an individual's observed and expected gene expression was used as described above. Genes with minimal expression ≥2\hat{ }−14.5 for MEBEMPs and ≥2\hat{ }−15.5 for CLPs across individuals were used. A LASSO model was trained using nested leave-one-out cross validation. For each left-out sample cross validation was performed on the remaining samples to select LASSO's □ parameter, a model was trained using the selected □ and a prediction was made on the left-out sample.

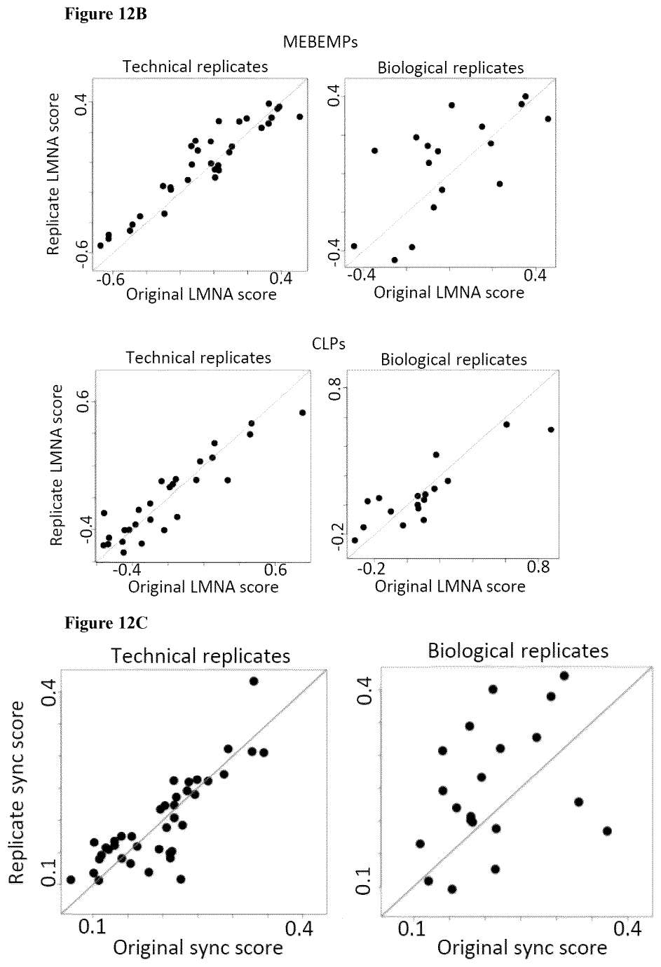

LMNA signature: The difference between an individual's observed and expected gene expression was used and this difference was correlated to ΔLMNA separately for MEBEMPs and CLPs. The MEBEMP and CLP correlation values were then summed and genes whose summed correlation was >0.7 were kept. Further, genes with high technical variance were removed, resulting in retaining 17 genes in the LMNA signature. To calculate individual LMNA signatures, the average value of these 17 genes in the observed-expected matrix of each individual for MEBEMPs and CLPs were selected separately. To plot FIG. 12A, the geometric mean of LMNA signature gene expression were calculated for each individual in each one of the 10 MPP/MEBEMP bins described earlier (FIG. 2D). GoT Analysis: GoT²⁰ performed on sample N122 allowed the marking of this individual's cells as wild-type or mutated. Due to the low VAF of N122's DNMT3A mutation, and in order to increase power, cells whose DNMT3A mutation status could not be determined by GoT were marked as wild-type cells. For FIG. 2G, N122's cells' distribution across cell states was examined.

Sync-score: The AVP signature was defined to include genes with high correlation (>0.6) to AVP across HSC, MPP and MEBEMP metacells, and the GATA1 signature to include those with high correlation (>0.7) to GATA1. Genes with mean relative expression >2\hat{ }−10 were filtered in these metacells, to preclude a small number of genes from dominating the signatures. All HSC, MPP, MEBEMP-E and MEBEMP-L cells was then scored by their fraction of its UMIs from the AVP and GATA1 signatures and all cells were partitioned into 20 equal-size bins of AVP signature expression and 20 equal-size bins of GATA1 signature expression. The sync-score is then defined as the fraction of cells in GATA1 bins 13 and above (upper two quintiles of GATA1) that are in AVP bins 9 and above (upper three quintiles of AVP expression).

To visualize the sync scores (FIG. 3I), this 20×20 bin matrix was normalized to sum to 1, the obtained matrix was smoothed by averaging cells using a running window of length 3, and log2 calculated.

Differential gene expression with respect to age and CBC: Differential expression was performed separately for MPP/MEBEMP and CLP cells as well as for males and females. The MPP and CLP-M matrices previously used to detect variant gene modules, were here as well. Individual gene expressions were correlated with age, max VAF of CH mutations and 20 CBC indices using Spearman correlation, and the correlation was tested for significance. p-values were FDR-corrected (Benjamini-Hochberg) for each label separately. For max VAF a Mann-Whitney test comparing individuals with and without detected mutations was additionally performed. Differential expression between males and females was performed using a Mann-Whitney test on the same expression matrices.

Patient scRNA-seq initial processing: All patient-including 10× libraries were multiplexed with additional healthy samples. These were processed using cellranger as described previously. Doublets were detected using Vireo and Souporcell and cells were assigned to individuals as described above. All patient data was sequenced on the Ultima platform and was corrected by downsampling and resampling of UMIs as described above. A metacell model was then created for each of 12 samples separately: 2 healthy individuals, 2 MDS patients (one of which was a del5q patient sampled twice-before and after treatment initiation), 3 CMML patients, 1 MDS/MPN overlap patient, 1 myelofibrosis patient and 2 AML patients. As previously described-cells with <500 UMIs, >20% mitochondrial gene expression, or with high expression of megakaryocyte genes were excluded from these models. The same set of ignored genes previously used for the healthy model were used and the target number of cells per metacell was set such that each metacell would have ˜300K UMIs.

Projection of disease data on the HSPC model: To project patient metacells on the healthy reference, patient (query) metacells were correlated with reference metacells. Due to sequencing depth variability, query metacells were first downsampled to 150K UMIs per metacell. The correlation was performed in log2 scale using variable genes from the reference. Query metacells were then annotated using the mode (most common cell state) of the 5 reference metacells they were most correlated to. Query metacells that mapped to CD34-negative reference metacells were discarded from downstream analyses. FIG. 4B shows the mean correlation between each query metacell and its 5 most correlated reference metacells and places each query metacell on the mean x and y coordinates of these metacells on the reference UMAP. FIGS. 4C-D are based on projection of single cells. Patient query cells were correlated to reference metacells using raw UMI vectors. FIG. 4C then shows the distribution of projected metacell annotations (determined by the mode of each query cell's 5 most correlated reference metacells). To create FIG. 4D, each query cell was assigned to the bin (as in FIG. 2D) most common among cells in the metacell to which it mapped. To create FIG. 4E, each patient's observed expression was calculated as the geometric mean expression across all his metacells annotated as either MPP/MEBEMP-E/MEBEMP-L. To calculate expected expression, the geometric mean expression of the 5 most correlated reference metacells of each query metacell were selected. Due to periodic systematic differences between query and reference samples, expected values were normalized by sorting genes based on their mean expression in the observed and expected and subtracting from the expected value a rolling mean of the difference between the expected and observed across 500 consecutive genes. Differentially expressed (DE) genes were defined as having ≥2-fold difference between observed and expected.

Karyotype analysis: To perform karyotype analysis, from each query metacell expression (normalized to sum to 1 and log2 taken) the geometric mean of its 5 most correlated reference metacells was subtracted (expression difference). Each chromosome was portioned into equal size binds, each containing ≥40 genes, and the median expression differences were computed across all genes in each bind. For this analysis, only genes with an average expression of at least 2\hat{ }−15.5 in either query or matched reference metacells were considered. This analysis provides metacell resolution karyotypes, as shown in FIG. 4F.

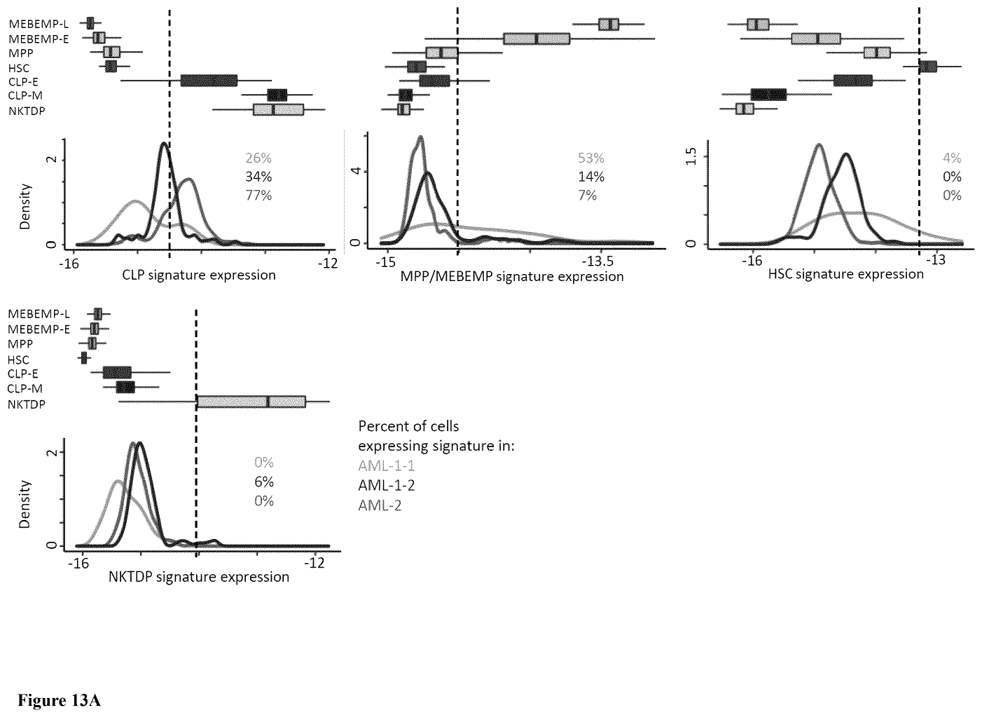

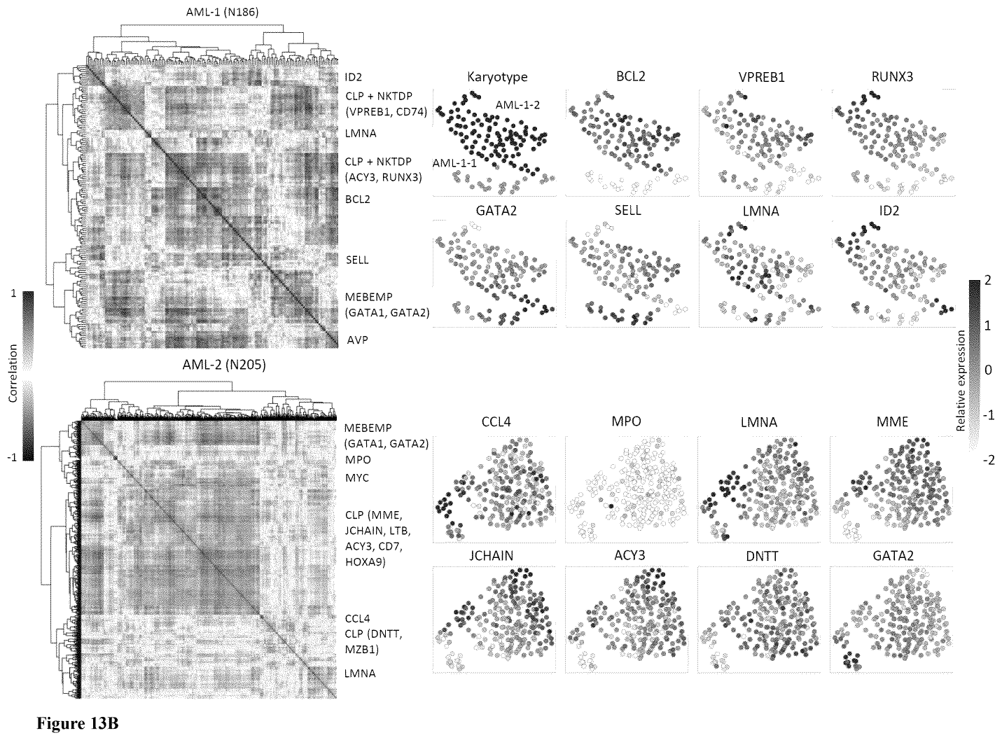

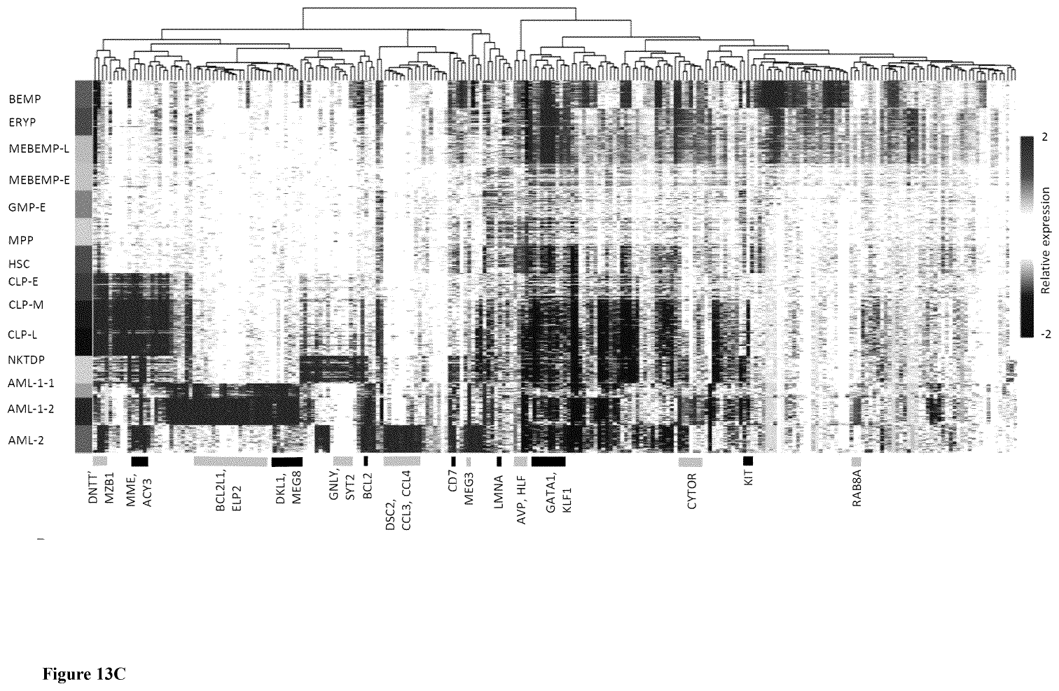

Profiling signatures in disease cases: To create FIG. 13, AML-1 (patient N186) metacells were separated into AML-1-1 and AML-1-2 by their karyotype. Lists of differentially (over)expressed genes in healthy HSC, MEBEMP, NKTDP and CLP metacells were created. Each AML metacell was scored by the geometric mean of all genes in each of these cell-state specific gene lists, and of the LMNA signature genes. State-specific expression thresholds were then set by observing the expression of each state-specific gene program across all reference metacells belonging to the relevant cell state (e.g., NKTDP metacells for the NKTDP gene program, see dashed lines in FIG. 13A). To discover de-novo gene programs in the AML samples (FIG. 13B), highly variable genes were selected from each AML metacell model, their correlation calculated across metacells, their correlation profiles clustered, and clusters with low mean correlation (<0.4) and genes with low mean correlation to their clusters (<0.4) filtered. Several genes of interest were manually added to the displayed correlation matrices (FIG. 13B). For the heatmap in FIG. 13C, state-specific genes were selected from the above-mentioned gene lists, as well as genes higher in AML-1/AML-2 compared to the reference across all cell states, and genes higher in AML-1-2 compared to AML-1-1 in MPP, MEBEMP-E and CLP-E metacells.

### Example 1: Universal Stem and Progenitor States Observed Across Humans in CD34+ Peripheral Blood

To evaluate interpersonal diversity in subtype distribution and regulation of circulating HSPCs (cHSPCs) from healthy humans, multiplexed scRNA-seq was combined with genotyping, and integrated clinical data. Multiplexing was resolved using SNPs identified in the 3′ UTR of cHSPC RNA facilitating precise matching of cells to individuals, and improving control for batch effects and doublets (FIG. 1A). Altogether, HSPCs from 79 males and 69 females between the ages of 23 and 91 years (median 61.5) were collected. Technical replicates were run on 39 individuals, and biological replicates on a follow-up cohort of 20 individuals (one year following their original sampling date). longitudinal CBCs were collected up to 5 years prior to scRNA-seq and deep targeted somatic mutation analysis was performed on DNA produced from their blood at sampling, to identify cases of CH. Following quality control and filtering, 846,762 single cell profiles were retained, which were normalized to control for sequencing-platform batch effects and which were combined to construct and annotate a metacell manifold model. 672,000 CD34+ single cells were retained for downstream analysis. These formed a rich repertoire of states, associated with cHSCs and their differentiation trajectories (FIG. 1B). The derived model recapitulated and deepened earlier characterization efforts of HSPC states from the bone marrow (BM), and while not fully reflecting BM dynamics, was compatible with scRNA-seq data previously produced suggesting that cHSPCs can serve as a highly accessible proxy for hematopoietic dynamics, both within and between individuals. One notable characteristic specific to cHSPCs, however, was the repression of cell cycle gene expression. Importantly, it was found that the cHSPC model was consistent among individuals. The median number of individuals contributing cells to each metacell was 84, and all metacells included cells from at least 47 individuals. Individual-specific differential expression was limited after controlling for each sample's cell distribution over the atlas states.

### Example 2: High Resolution Circulating HSC Map Shows HLF, GATA3, HOXB5 and TLE4 as Distinct HSC TFs

One of the hallmarks of this cHSPC model is a distinct HSC state that is transcriptionally linked with two major differentiation gradients: the first representing a continuum of common lymphoid progenitor (CLP) programs; the second, and more common branch, representing multipotent progenitor (MPP) states and their differentiation toward granulocyte-monocyte progenitors (GMPs), erythrocyte progenitors (ERYPs) and basophil/eosinophil/mast progenitors (BEMPs). Technical limitations of cell disassociation in scRNA-seq prevented precise megakaryocyte program modeling. Therefore, states at the base of this trajectory were annotated as megakaryocyte/erythrocyte/basophil/eosinophil/mast progenitors (MEBEMP) as these are also presumed to be the cells of origin of megakaryocytes.

Early HSCs are marked by high AVP and HLF expression and were previously shown to represent a rare cell population with self-renewal capacity in BM and cord blood. This model included data on ˜14,440 HLF/AVP HSCs that could be matched with cells from independent BM atlases, suggesting that under steady-state, HSCs with potential self-renewal capacity are present in the peripheral blood. Together with HLF and AVP, 14 genes were discovered that were expressed at least 1.75-fold higher in HSCs as compared to their two immediate differentiation branches. Several transcription factors (TFs) enriched in HSCs were specifically identified, including the genes HOXB5, TLE4 and GATA3 (FIG. 1C). It was noted that while the HSC state is defined by unique markers that are symmetrically down-regulated upon exit of the CLP and MEBEMP trajectories (FIG. 1C), it also expresses several lineage-specific regulators at intermediate levels, which are bifurcating anti-symmetrically upon exit from the HSC state to the CLP and MEBEMP trajectories (FIG. 1D). This may suggest that the multipotent capacity of HSCs is associated with intermediate expression of multiple regulators which is resolved with differentiation.

### Example 3: NK-T-Dendritic and Basophil-Eosinophil-Mast Progenitors are Enriched in Circulating HSPCs

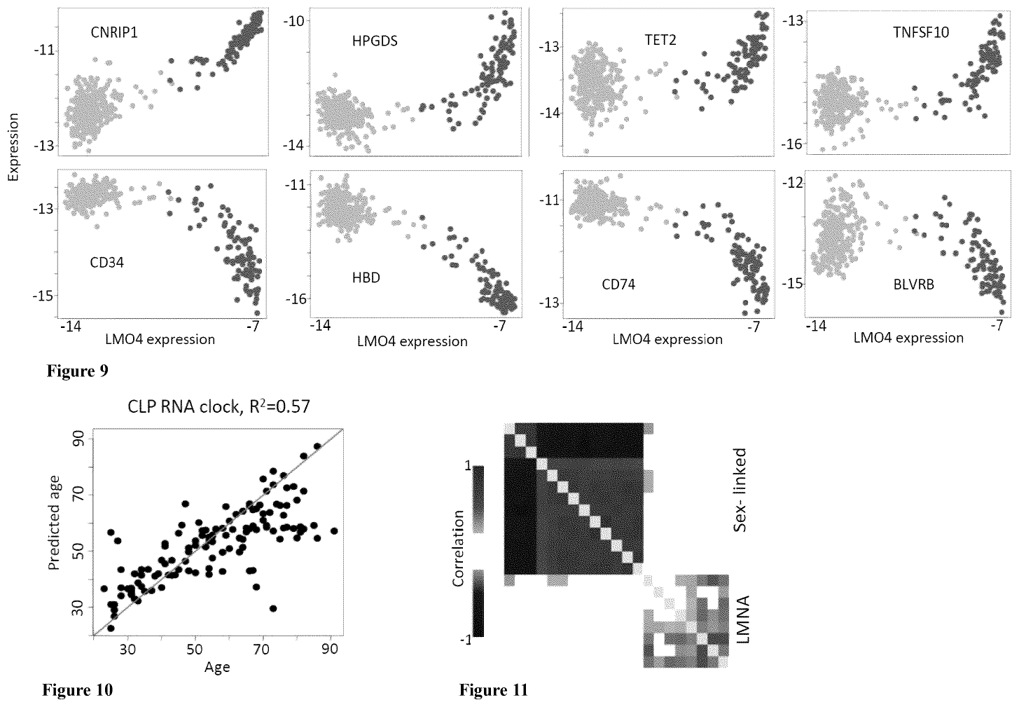

The cHSPC atlas was enriched for basophil-eosinophil-mast progenitors (BEMP), mapped as one possible terminus of the HSC differentiation. While classical studies linked these cells with a granulocyte/monocyte progenitor (GMP) origin, more recent studies suggested these emerge, at least in part, from erythroid progenitors in mice and humans. This analysis allowed for focusing on a small population of metacells linking BEMPs with their MEBEMP-L precursors (FIG. 1E). This highlighted TFs (FIG. 1F) and other factors (FIG. 9) positively or negatively regulated in this postulated early stage of BEMP specification. Another rare HSPC population that could now be focused on included lymphoid states with high ACY3 expression and intermediate-to-low DNTT levels, a combination rarely found in the human BM but present in peripheral blood. Interestingly, co-variation of key T cell regulators was observed within this population, but also anti-correlation of these factors with some hallmarks of a dendritic cell (DC) program. This can be demonstrated by comparison of TCF7 and IRF8 expression (FIG. 1G), and the matching TCF7-coupled dynamics of CD7, MAF, and IL7R, or IRF8-coupled dynamics of the myeloid TF SPI1 (PU.1) and multiple MHC-II genes (FIG. 1H). This subpopulation was therefore termed NK/T/DC progenitors (NKTDP). To summarize, the map of circulating HSPCs showed a rich spectrum of differentiation trajectories and progenitor states that refined previous analyses and, a remarkable universality of states, which provided an opportunity for deciphering inter-individual hematopoietic variability.

### Example 4: Inter-Individual Variation in cHSPC Stemness and in Lymphoid/Myeloid Differentiation Bias

To study inter-individual cHSPC variation, first, individual-specific cell state compositions were looked at. This was performed by quantifying cell state relative frequencies within each individual's single-cell ensemble (FIG. 2A). These frequencies varied extensively (FIG. 2B). For example, HSCs and CLP-Ms, representing 2.4% and 12.6% of the CD34+ population on average, respectively, showed standard deviations of 1.0% and 6.8%, respectively. The abundant MPP and MEBEMP-E states (mean frequency of 20.7% and 37.6%, respectively) showed smaller relative variation (SD 4.9% and 5.8%, respectively). To analyze the stability of cell state frequencies over time and sampling instances, 20 individuals were re-sampled one year following their original sampling date. Both lymphoid progenitor frequencies (CLP-M, CLP-L, NKTDP), and MEBEMP (MEBEMP-E, MEBEMP-L, ERYP, BEMP) frequencies were stable within the same individual across time (FIG. 2C).

To analyze composition in higher resolution, each individual's enrichment was profiled over the MEBEMP and CLP trajectories. Clustering of these enrichment profiles yielded six archetypes of cHSPC composition within the healthy population (classes I-VI) (FIG. 2D). These were composed of individuals with relative lymphoid enrichment (class I-III) or depletion (class V-VI) further subdivided by a stemness gradient, enriched in classes II, IV and VI, and depleted in class I, III and V. Analysis of technical and biological replicates confirmed this variation to be robust and patient specific. To summarize, the instant analysis provides the first cHSPC subpopulation normal reference range (FIG. 2B), characterized by extensive variation among healthy individuals, and show these compositional differences are a true individual characteristic, with potential clinical implications.

### Example 5: Circulating HSPC Frequencies Correlate with CBCs and CH

Analysis of CBC correlations with the instant single-cell atlas enhanced previous findings on the inter-individual variation in cHSPC compositions. All CBC correlation analyses were performed using median values for each blood count parameter over 5 years preceding scRNA-seq. The mean and median number of blood counts per individual during this 5-year period were 8, and 6 respectively. A significant positive correlation (P<0.01_ was observed between PB mature lymphocyte percentages and CLP frequencies (FIG. 2E, left). Given the very high variability in female red blood cell (RBC) count and size during young adulthood (with menarche and pregnancy effects) as well as during prolonged periods of perimenopause, RBC indices, including RBC, hematocrit (HCT), mean corpuscular volume (MCV), and RDW, were analyzed separately in males and females. A significant negative correlation (P<0.02) was observed between CLP frequencies and HCT (males, FIG. 2E—middle). As well as a significant positive correlation (P<0.01) between increased RDW—a cHSPC myeloid bias—and a relative CLP depletion (males, FIG. 2E—right).

Previous work correlated increased RDW with high risk for CH and predisposition to acute myeloid leukemia (AML). It is demonstrated that low CLP frequencies are associated with CH (two-sided Mann-Whitney test; FIG. 2F) and this further enhances this observation by performing Genotyping of Transcriptomes on one of the DNMT3A R882 cases, identifying a lower fraction of CLP cells in the mutant clone (P<0.005, Fisher's exact test, FIG. 2G). To further explore this association, a cohort of 18147 healthy individuals for whom both longitudinal CBCs and DNA were available were studied. 602 individuals with a high RDW (>15%, not meeting minimal criteria for myelodysplastic syndrome (MDS) diagnosis) and 602 age and sex matched normal RDW controls were identified. Deep targeted sequencing was performed to identify pre-leukemic mutations (pLMs) on both high-RDW individuals and controls and a significant enrichment of CH+ cases in the high RDW group was found (Fisher's exact test P-value<0.002, FIG. 2H). Altogether, the data demonstrate a 3-way linkage between decreased CLP frequencies, a high RDW, and CH.

### Example 6: Age-Related Myeloid Bias is Predominantly Observed in Males

Blood aging is a complex and multi-factorial process, likely driven by intrinsic factors such as pre-leukemic mutations, and extrinsic effects, such as cytokine and hormonal changes. In order to decouple these factors as much as possible, age-related changes in cHSPC populations were studied in individuals without CH mutations. Analysis of age-linked compositional changes in cHSPCs within this group showed a remarkable increase in myeloid (MEBEMP) to lymphoid (CLP) ratios in males (when comparing <50 to >60-year-old individuals, FIG. 3A). This effect was not significant in females. In this regard it is important to note that a decline in lymphocyte counts can be observed in both elderly males and females, however it appears in females at an older age. Interestingly, females exhibit a surge in lymphocyte counts immediately following menopause, contributing to this delay in lymphocyte decline. Within the MEBEMP differentiation trajectory, aging was correlated with over-representation of more differentiated states, once again only in males (FIG. 3B). Of note, the frequencies of cHSCs did not significantly change with age (FIG. 3C). While previous studies suggested aging is linked with an increase in HSC frequencies, such increase was not observed with the restrictive definitions employed here, as well as when determined from CD34+ PB HSPC frequencies in a recent cohort of 1000 healthy individuals undergoing PBMC scRNA-seq (FIG. 3D). The sex-specific correlation between age and cHSPC myeloid bias highlights the role of such non-intrinsic effects on this classical hallmark of blood aging.

### Example 7: Composition-Controlled HSPC Expression Correlates with Age

As shown above, an individual's cHSPC composition provides an initial blueprint of hematopoietic dynamics along the stemness and CLP/MEBEMP axes. Further analysis of transcriptional variation could now be carried out, while controlling for the dominant effect of cHSPC composition, in order to characterize additional gene expression signatures that could distinguish between individuals. Composition-controlled individual expression profiles showed high information content when correlated with age, enabling age prediction based on normalized expression alone (FIGS. 3E, 10). Next, a search for gene groups (signatures) that co-variate between individuals was performed, filtering out sex-linked signatures and those showing strong batch effects. The most prominent of these signatures included Lamin-A (LMNA) as well as annexin A1 (ANXA1), AHNAK nucleoprotein (AHNAK), myeloid associated differentiation marker (MYADM), tetraspanin 2 (TSPAN2), and vimentin (VIM), among others (FIGS. 3F, 11). Individual LMNA signature expression varied across a range of more than 2-fold (FIG. 12A), exhibiting high expression variability in HSCs and early myeloid and lymphoid cell states, and a homogeneously low expression in late MEBEMPs and CLPs. Individual LMNA signature expression was consistent in myeloid and lymphoid cell states (FIG. 3G) and was stable in a follow-up cohort (FIG. 12B). Interestingly, an age-linked increase in LMNA signature expression was observed in lymphoid, but not myeloid, cHSPCs (FIG. 3H). Taken together, this shows that in addition to the accumulation of pLMs in HSPCs, aging is strongly linked with changes in the distribution of progenitor cell states in the PB, and with significant differences in the expression of certain gene signatures.

### Example 8: Rapid Repression of Stemness Signatures in MEBEMPs is Linked with Lower Red Cell Counts and Higher Red Cell Volumes

The differentiation of HSPCs toward MEBEMP and CLP fates involves coordinated activation of specific transcriptional programs that were generally universal among individuals. Yet, the screen for individual-specific gene signatures suggested that individuals differed in the way they synchronized the opposing effects of these stemness and differentiation programs. To quantify this variation, AVP (stemness) and GATA1 (MEBEMP differentiation) signatures were compared on a 20×20 bin expression matrix (FIG. 3I). While most individuals displayed dynamics close to the diagonal line (individuals N16, and N86 for example), following the typical transition from stemness to differentiation, some individuals deviated from the diagonal, indicating skewed synchronization between the AVP and GATA1 signatures. This deviation (i.e. off-diagonal frequency) was quantified using a new synchronization-score. This facilitated the identification of individuals with sync-scores as low as 0.12 (e.g., N122 and N172, FIG. 3I, top), indicating delayed activation of GATA1 relative to AVP repression. Namely, while these individuals rapidly reduce their AVP expression, their increase in GATA1 and GATA1-related genes is delayed. In contrast, individuals exhibiting a high sync-score (e.g., N98 and N121, for example, FIG. 3I, bottom), show early activation of GATA1 expression, which precedes AVP repression. Stability of the sync-score was detected in a follow-up cohort (FIG. 12C). Inter-individual sync score variability was positively correlated with RBC levels, and consistently anti-correlated with MCV in males (P<0.01 (Spearman) for both RBC and MCV; FIG. 3J). Analysis of the correlation between individual sync-scores and cHSPC compositions in males demonstrated a negative correlation with ERYPs and BEMPs (FIG. 3K). To summarize, there was demonstrated variation in the coordination of stemness and MEBEMP differentiation programs that is correlated with red blood cell counts and volumes.

### Example 9: Age-Related Perturbation of HSPC Composition and Transcriptional Signatures

Aging in the blood represents a complex and multi-factorial process that is likely driven by intrinsic hematopoietic effects (e.g., pre-malignant mutations) and extrinsic physiological effects (e.g., hormonal changes). We therefore anticipated multiple properties to define a multi-layered age-HSPC correlation. We first tested the association between HSPC compositions and age and did not observe an apparent directional increase or decrease in HSPC sub-types with aging. We did demonstrate an increase in the variance of cell state frequencies, with a significantly higher variance above the age of 65 (p<0.01). To quantify each individual's deviation from expected cell state frequencies, we computed an HSPC composition bias score, which significantly increased with age (FIG. 4A, p<0.02, test for Spearman's rho). This supported the notion of multiple age-related processes that perturb the highly homogeneous and robust HSPC landscape seen in young adults.

We used several HSPC signatures to further study inter-individual variation in aged hematopoiesis, including the LMNA and sync signatures described above, as well as an S-phase signature, quantifying expression of S-phase related cell-cycle genes, previously shown to have high inter-individual composition-normalized gene expression correlation (FIG. 3B). The S-phase signature was robust in the follow-up cohort, supporting its role in characterizing an individual quality rather than a transient effect. Circulating HSPCs did not generally express S-phase transcriptional signatures, in contrast to their bone-marrow counterparts (FIG. 4B). However, weak, but significant, expression of DNA replication genes was observed in the late MEBEMP trajectory of some individuals, with a strong positive association with age (FIG. 4C, p<0.04, test for Spearman's rho). Comparison of S-phase signatures to HSPC composition bias scores suggested the two increased independently with age (FIG. 4D). In contrast, increased HSPC bias scores could be associated with lower LMNA signatures (FIG. 4E), strengthening the association between CH and low LMNA expression. Sync scores were not directly correlated with age (FIG. 4F), despite their associations with RBC and MCV as described above.

Case studies of individuals with highly abnormal HSPC distributions, and integration of these with clinical markers and mutation profiling illustrate the multi-modal nature of hematopoietic aging. Individual #151, an 80yo MDS-diagnosed male, defined by a TET2/DNMT3A/CBL clone with high variant allele frequency (VAF; TET2 VAF=70%) and exhibiting high RDW anemia, shows extreme HSPC bias, a low LMNA signature and a high S-phase signature (FIG. 4G). Individual #98, a 69yo male, represented another distinct behavior, with polycythemia, a high sync signature and high RDW. Taken together, the analysis of HSPC composition and transcriptional signatures provided insights to the various mechanisms that drive hematopoietic aging. In particular, our analysis separates the spectrum of effects associated with CH, from those associated with changes in HSPC regulation and differentiation. High resolution characterization of these effects enables the analysis of patients with blood malignancies at high molecular depth.

### Example 10: Using the cHSPC Atlas for Mapping, Dissecting and Annotating Myeloid Malignancies

Diagnosis of myeloid malignancies requires the identification of clonal markers (mutations or structural variants) and the detection and quantification of blasts by microscopy and flow cytometry. In FIG. 5A there is shown a new stepwise approach for analysis of myeloid disorders based on sampling of cHSPCs and comparison to their compositions, normalized expression and copy number variations (karyotyping) to the new normal reference. As a proof of concept, sampled cells from 2 healthy individuals, 3 chronic myelomonocytic leukemia (CMML) patients, 2 MDS patients, 1 MDS/myeloproliferative neoplasm (MPN) overlap patient and 1 myelofibrosis (MF) patients were analyzed. Further, two AML cases were sampled and analyzed to demonstrate how acute disease is manifested when projected over the cHSPC model. Projection of patient metacells showed a high gene expression correlation between metacell pairs (FIG. 5B, color coded dots), for all pathological cases except for the AMLs. Cell state compositions were, however, skewed in all pathological cases (FIG. 5C-5D), but not for the healthy controls. All patient samples showed a remarkable reduction in CLP populations (FIG. 5C-5D). Two of the CMML patients (N192, N235) demonstrated highly abnormal enrichment of specific (basophil and myeloid) cell states, while the rest showed a relatively balanced distribution over the MEBEMP differentiation spectrum (with slight enrichment of stem states). Compositional-controlled gene expression comparisons of patient samples to the normal model identified specific genes that were recurrently induced or repressed in disease (FIG. 5E). While both healthy individuals and the treated MDS case showed a minimal number of DE genes, all leukemic cases exhibited a substantial increase in the number of DE genes when compared to the healthy population model (FIG. 5E).

Detection of karyotypic abnormalities based on gene expression dosage effects, previously suggested and implemented in several tools, can be readily implemented on cHSPCs, as shown in FIG. 5F, where clear chromosomal aberrations are observed in the two AML cases analyzed. Further, deletion of the long arm of chromosome 5, del(5q) was detected in one of the MDS samples with complete cytogenic remission following lenalidomide treatment (N211A & N211B, FIG. 5F). To conclude, the atlas of normal cHSPC states presented herein enables high resolution characterization of myeloid disorders, based on their cell states, compositional-controlled transcriptional variance, and abnormal karyotypes.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A non-invasive method of detecting pathology of the bone marrow in a subject in need thereof, the method comprising:
a. receiving a subject cellular dataset based on single cell RNA sequencing (scRNA-seq) of CD34 positive cells from peripheral blood of said subject; and
b. analyzing said received subject cellular dataset in relation to a control dataset comprising a plurality of cellular datasets wherein each cellular dataset of said plurality is based on scRNA-seq of CD34 positive cells from peripheral blood of a healthy subject, wherein a deviation of said subject cellular dataset from said control dataset indicates a bone marrow pathology, optionally wherein said analyzing comprises producing a feature vector representing deviation of the subject's cellular data from the control cellular data;
thereby detecting pathology of the bone marrow.

2. The method of claim 1, wherein said cellular dataset comprises statistical data of the totality of CD34 positive cells in a peripheral blood sample.

3. The method of claim 1, wherein said analyzing comprises applying a trained machine learning model to said received dataset, wherein said machine learning model is trained on a training set comprising said plurality of cellular datasets and wherein said machine learning model classifies said subject's bone marrow as being a healthy or not and wherein said training set further comprises cellular datasets based on scRNA-seq of CD34 positive cells from peripheral blood of subjects suffering from pathology of the bone marrow and labels indicating a cellular dataset is from a healthy subject or a subject with pathology of the bone marrow; and wherein said machine learning model classifies said subject as being heathy or suffering from a pathology of the bone marrow.

4. The method of claim 1, wherein said analyzing comprises producing a feature vector representing deviation of the subject's cellular data from the control cellular data and applying a trained machine learning model to said feature vector, wherein said machine learning model is trained on a training set comprising: feature vectors from healthy subjects and subjects suffering from pathology of the bone marrow and labels indicating a feature vector is from a healthy subject or a subject with pathology of the bone marrow; and wherein said machine learning model classifies said subject as being heathy or suffering from a pathology of the bone marrow.

5. The method of claim 1, wherein said analyzing comprises applying a trained machine learning model to a parameter extracted from said cellular dataset, wherein said machine learning model is trained on a training set comprising: said parameter extracted from cellular datasets of healthy subjects and optionally subjects suffering from a bone marrow pathology and wherein said machine learning model classifies said subject as being a healthy subject or not.

6. The method of claim 1, wherein said cellular dataset is a metacell model of the totality of CD34 positive cells in a peripheral blood sample.

7. The method of claim 1, wherein said pathology of the bone marrow is selected from myelodysplastic syndrome (MDS), Chronic myelomonocytic leukemia (CMML), Acute myeloid leukemia (AML), polycythemia vera (PV), essential thrombocythemia (ET), Mastocytosis, chronic eosinophilic leukemia, myelofibrosis (MF), acute lymphoblastic leukemia (ALL), acute leukemia of ambiguous lineage, multiple myeloma (MM), myeloproliferative neoplasm (MPN) and blastic plasmacytoid dendritic cell leukemia.

8. The method of claim 7, wherein said method is at least one of:
a. a method of detecting MDS and wherein deviation in the frequency of erythrocyte progenitor cells (ERYP), basophil/eosinophil/mast progenitor cells (BEMP), and/or megakaryocyte/erythrocyte/basophil/eosinophil/mast progenitor cells (MEBEMP) indicates the presences of MDS;
b. a method of detecting MDS and wherein a decrease in the frequence of CLP, NKTDP or both as compared to healthy subjects is indicative of MDS;
c. a method of detecting CMML and wherein deviation in the frequency of early granulocyte-monocyte progenitor cells (GMP-E) indicates the presence of CMML; and
d. a method of detecting AML and wherein deviation in the frequency of common lymphoid progenitor cells (CLP) and/or natural killer/T/dendritic cell progenitor cells (NKTDP) indicates the presence of AML.

9. The method of claim 8, wherein deviation in the frequency of CLPs is also indicative of MDS and wherein said deviation is lower levels of said CLPs than is present in said healthy subjects.

10. The method of claim 8, wherein deviation in the frequency of CLPs is also indicative of CMML, MF or MPN and wherein said deviation is lower levels of said CLPs than is present in said healthy subjects.

11. The method of claim 8, wherein a decrease in the frequency of both CLP and NKTDP as compared to healthy subjects is indicative of MDS.

12. The method of claim 1, wherein said pathology of the bone marrow comprises an increased percentage of blasts, wherein deviation is an increase and wherein a deviation in the frequency of early common lymphoid progenitor cells (CLP-E) indicates the presence of an increased percentage of blasts.

13. The method of claim 1, further comprising administering at least one therapeutic agent to a subject determined to suffer from a bone marrow pathology.

14. A non-invasive method of predicting the percentage of blasts in the bone marrow of a subject in need thereof, the method comprising:
I. receiving a measure of the CLP-E cells in the peripheral blood of said subject wherein said measure is proportional to the percentage of blasts in the bone marrow of said subject, and optionally analyzing said received measure in relation to a control dataset comprising a plurality of measures of CLP-E cells in the peripheral blood of healthy subjects and subjects suffering from pathology of the bone marrow, wherein the percentage of blasts in the bone marrow is known for each subject of said control dataset; or II. a) receiving a subject cellular dataset based on single cell RNA sequencing (scRNA-seq) of CD34 positive cells from peripheral blood of said subject; and;
b) applying a trained machine learning model to said received dataset, wherein said machine learning model is trained on a training set comprising a plurality of cellular datasets wherein each cellular dataset of said plurality is based on scRNA-seq of CD34 positive cells from peripheral blood of a control subject and labels indicating the percentage of blasts in the bone marrow of said control subjects that provided each cellular dataset of said plurality of cellular datasets; and wherein said machine learning model ;outputs a predicted percentage of blasts in the bone marrow of said subject .thereby predicting the percentage of blasts in the bone marrow of a subject

15. The method of claim 14, wherein said subject suffers from leukemia and said control subjects comprise subjects suffering from leukemia and non-leukemic subjects.

16. The method of claim 14, wherein said cellular dataset is a metacell model of the totality of CD34 positive cells in a peripheral blood sample.

17. The method of claim 8, wherein said cellular data set is a metacell model and is produced by a method comprising:
a. receiving a peripheral blood sample from a subject;
b. isolating CD34 positive hematopoietic stem and progenitor cells (HSPCs) from said peripheral blood sample;
c. performing scRNA-seq of said isolated HSPCs to produce a transcriptome for each isolated HSPC; and
d. producing a metacell model of said HSPCs based on their transcriptomes wherein a metacell is a cluster of cells with a similar transcriptome.

18. The method of claim 14, wherein a cellular dataset comprises groupings of cells into cell types that share a common differentiation within the HSPC spectrum of differentiation and wherein said cell types are selected from: BEMP, ERYP, MEBEMP-L, MEBEMP-E, GMP-E, multipotent progenitor cells (MPP), hematopoietic stem cells (HSC), CLP-E, CLP-M, CLP-L and NKTDP.

19. The method of claim 14, wherein said method is a method of detecting MDS and/or leukemia and wherein a percentage of blasts above a predetermined threshold indicates said subject suffers from MDS and/or leukemia and administering to a subject suffering from MDS and/or leukemia at least one anticancer therapy.

20. A non-invasive method of calculating a Molecular International Prognostic Scoring System (IPSS-M) risk score for a subject suffering from a bone marrow malignancy, the method comprising:
a. predicting the percentage of blasts in the bone marrow of said subject by a method of claim 14;
b. detecting the presence of bone marrow mutations and karyotype abnormalities based on scRNA-seq reads from CD34 positive cells from peripheral blood of said subject;
c. receiving hemoglobin levels, and platelet counts in peripheral blood from said subject;
d. calculating said IPSS-M risk score based on said predicted blast percentage, detected mutations and karyotyping and received hemoglobin levels and platelet counts; and
e. administering to said subject a treatment regimen based on said IPSS-M risk score, where in a subject with a higher score is administered a more intense treatment regimen and a subject with a lower score is administered a reduced treatment regimen;
thereby calculating an IPSS-M risk score.
